(19) United States
(12) Patent Application Publication
(10) Pub. No.: US 2005/0203065 A1
(43) Pub. Date: Sep. 15, 2005

(54) Method of treating ischemia reperfusion injury using adenosine receptor antagonists

(76) Inventors: Glenn J. Smits, Hudson, WI (US); Xiaowei Jin, Cambridge, MA (US); Garrett J. Gross, Elm Grove, WI (US); John Auchampach, Wauwatosa, WI (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/006,022

(22) Filed: Dec. 6, 2004

Related U.S. Application Data
(63) Continuation of application No. PCT/US03/18695, filed on Jun. 12, 2003.
(60) Provisional application No. 60/388,680, filed on Jun. 12, 2002.

(51) Int. Cl. A61K 31/522; A61K 31/675

(52) U.S. Cl. 514/81; 514/263.34; 514/263.24

(57) ABSTRACT

Methods useful for preventing, limiting, or treating ischemia reperfusion injury in a mammal are disclosed. More particularly, this invention relates to administering A_{2b} adenosine receptor antagonists to prevent, limit or treat ischemia reperfusion injury.

### TECHNICAL FIELD OF THE INVENTION

This invention relates to cardiology, medicinal chemistry and pharmacology. More particularly, it relates to A_{2b} adenosine receptor antagonists and preventing or treating ischemia reperfusion injury.

### BACKGROUND OF THE INVENTION

The cessation of blood flow and oxygen delivery to a tissue induces a condition known as ischemia. Substantial reductions of oxygen delivery induce a condition known as hypoxia. Both ischemia and hypoxia, if prolonged, can result in the loss of function in the tissue and even cell death. There are numerous conditions, both natural and iatrogenic, that cause ischemia and hypoxia including, but not limited to, occlusive vascular disease, coronary thrombosis, cerebrovascular thrombosis, aneurysm rupture, general hemorrhage, crush injury, sepsis, severe cutaneous burns, vasculo-occlusive surgical techniques (such as spinal ischemia during thoracoabdominal aneurysm surgery), cardiopulmonary bypass procedures, organ transplantation, cardiopulmonary collapse (sudden cardiac death), and suffocation.

Conventional treatment for ischemia and hypokia is to restore blood flow and oxygen delivery to normal levels, either by increasing general oxygenation or by removing the cause of the vascular blockage. Restoration of blood flow results in improved outcomes when compared to situations wherein ischemia or hypoxia are maintained for longer periods of time. However, it is w ell recognized that the restoration of bloodflow and oxygen delivery can cause additional cell death and loss of function independent of the damage caused by ischemia or hypoxia. This additional damage induced by the restoration of blood flow and oxygen delivery is known as reperfusion injury. The paradoxical tissue damage caused by reperfusion injury appears to be similar to an acute inflammatory condition, resulting from the adherence of inflammatory cells to the reperfused tissues, activation of these inflammatory cells and the subsequent generation of free radicals (Granger et al. Ann. Rev. Physiol., 57, 311-332, (1995)). The generation of free radicals and other cytotoxic biomolecules within reperfused tissue can induce cell death by either necrosis or by activation of the apoptosis pathway.

Adenosine is an intracellular and extracellular messenger generated by all cells in the body. It is also generated extracellularly by enzymatic conversion. Ischemic and hypoxic tissues generate increased quantities of adenosine, via the breakdown of adenosine triphosphate (ATP) during energy consumption. These adenosine receptors are divided into four known subtypes (i.e., A₁, A₂ₐ, A_{2b} and A₃) based on their relative affinity for various adenosine receptor ligands and by sequence analysis of genes encoding these receptors. The activation of each of the subtypes elicits unique and sometimes opposing effects.

Three of the four adenosine receptor subtypes are known to influence the function of inflammatory cells during reperfusion injury. Activation of A₂₈ adenosine receptors has been shown to suppress the release of oxygen free radicals from stimulated neutrophils, to reduce the adherence of neutrophils to vascular endothelium, and to suppress neutrophilic release of TNF and LTB₄ (see, e.g., Cronstein et al., J. Immunology, 148, pp. 2201-2206 (1992); Thiel et al., (1995) J. Lab. Clin. Med., 126, pp. 275-282; Krump et al., J. Exp. Med., 186, pp. 1401-6(1997)).

In contrast to the anti-inflammatory effects of A₂ₐ adenosine receptor activation, activation of A₁ receptors has been shown to promote chemotaxis and phagocytosis by stimulated neutrophils, (see, e.g., Cronstein et al. (1992), supra; Salmon et al., J. Immunology 145, pp. 2235-2240. (1990)) and to promote monocyte differentiation into multinucleated giant cells (Merrill et al., Arth. Rheum., 40, pp. 1308-1315 (1997)). Moreover, activation of A₁ receptors on vascular endothelial cells promotes inflammation and tissue injury in a model of reperfusion injury of the heart (Becker et al., Pharm. Pharmacol. Letters, 2, pp. 8-11 (1992); Schwartz et al., J. Mol. Cell. Cardiol., 25, pp. 927-938 (1993); Zahler et al., Cardiovascular Res., 28, pp. 1366-1372 (1994); and Forman et al., J. Pharmacol. Exp. Ther., 292(3), pp. 929-38 (2000)).

Activation of the A_{2b} receptor can also lead to pro-inflammatory activities such as an increased production of IL-6 (Sitaraman et al., J. Clin. Invest., 107, pp. 861-9 (2001), and mast cell degranulation, a hallmark of local inflammation (Linden et al., Life Sci., 62, pp. 1519-24 (1998); and Auchampach et al., Mol. Pharmacol., 52, 846-60 (1997)). In addition, activation of A_{2b} receptors in vascular smooth muscle cells leads to loss of cells via direct stimulation of apoptosis (Peyot et al., Circ. Res., 86, pp. 76-85 (2000)).

Current treatments for ischemia-reperfusion injury only adequately treat the ischemic damage by restoring blood flow and oxygenation. However, the damage caused by reperfusion injury is generally under-treated. Investigational treatments for ischemia-reperfusion include the use of adenosine and adenosine analogs as well as inhibition of the sodium-calcium exchange pump on the ischemic myocytes. These therapies, however, are not sufficiently adequate. For example; the use of adenosine and adenosine analogs is burdened by the undesirable effects of depressor activity and bradycardia. Similarly, inhibition of the sodium-calcium exchange pump on the ischemic myocytes is inadequate because it does not prevent or treat the imflammatory conditions or the direct stimulation of apoptosis. Thus, there remains a need for new pharmaceutically acceptable compounds and compositions for preventing limiting or treating ischemia reperfusion injury.

### SUMMARY OF THE INVENTION

Applicants have solved the above problem by discovering that A_{2b} adenosine receptor antagonists are capable of preventing, limiting or treating ischemia reperfusion injury. The invention relates to a method for preventing, limiting or treating ischemia reperfusion injury in a mammal that has undergone an ischemic event or in which an ischemic event is imminent using A_{2b} adenosine receptor antagonists. The compounds useful in the methods of this invention exert their desirable effects through specifically antagonizing or blocking the A_{2b} adenosine receptor.

In some embodiments, the methods of this invention comprise administering to a patient a therapeutically effective or prophylactically effective amount of an A_{2b} adenosine receptor within ten: days before or after the ischemic event.

In some embodiments of the invention, the A_{2b} adenosine receptor antagonist is a compound of formula (I) 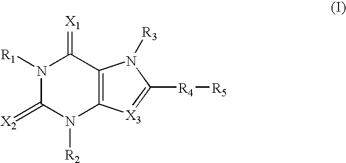
or a pharmaceutically acceptable salt or N-oxide thereof, wherein: each of R₁, R₂, and R₃, independently, is:
a) hydrogen;
b) C₁₋₆ alkyl, C₂₋₆ alkenyl, or C₂₋₆ alkynyl; wherein said alkyl, alkenyl, or alkynyl is either unsubstituted or substituted with one or more substituents selected from the group consisting of hydroxy, alkoxy, amino, monoalkylamino, dialkylamino, cycloalkyl, aryl, heterocyclyl, aralkyl, heterocyclylalkyl, acylamino, alkylaminocarbonyl, alkylsulfonylamino, and alkylaminosulfonyl;
c) substituted or unsubstituted aryl; or
d) substituted or unsubstituted heterocyclyl;
R₄ is a single bond, —O—, —(CH₂)₁₋₃—, —O(CH₂)₁₋₂—, —CH₂OCH₂—, (CH₂)₁₋₂O—, —CH═CHCH₂—, —CH═CH—, or —CH₂CH═CH—;
R₅ is:
(a) phenyl, or
(b) a bicyclic or tricyclic group selected from the group consisting of: 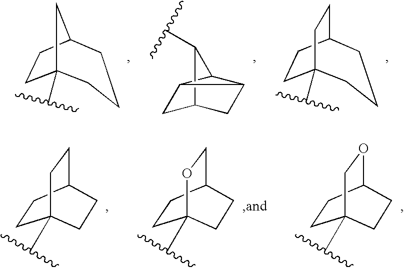
   wherein the phenyl, bicyclic, or tricyclic group is either unsubstituted or substituted with one or more Rₐ groups, which is selected from the group consisting of:
   (a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino) (R_{b}) acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phosphono, substituted or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
   (b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl(alkyl)amino-, R_{b}-alkyl(alkyl)carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl;
R_{b} is selected from the group consisting of —COOH, —C(CF₃)₂OH, —CONHNHSO₂CF₃, —CONHOR_{c}, —CONHSO₂R_{c}, —CONHSO₂NHR_{c}, —C(OH)RCPO₃H₂, —NHCOCF₃, —NHCONHSO₂R_{c}, —NHPO₃H₂, —NHSO₂R_{c}, —NHSO₂NHCOR_{c}, —OPO₃H₂, —OSO₃H, —PO(OH)R_{c}, —PO₃H₂, —SO₃H, —SO₂NHR_{c}, —SO₃NHCOR_{c}, —SO₃NHCONHCO₂R_{c}, and the following: 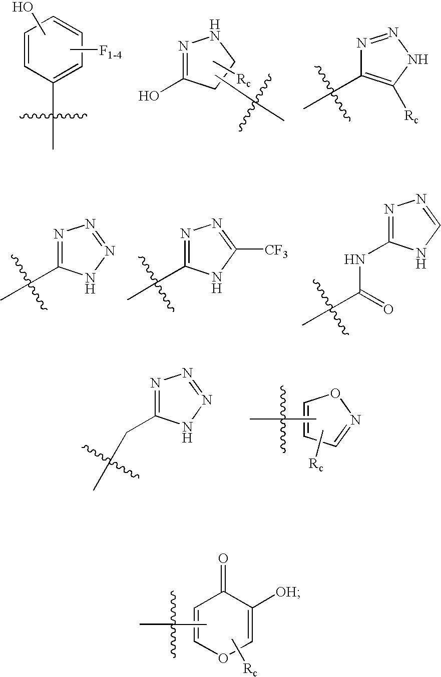
R_{c} is selected from the group consisting of hydrogen, —C₁₋₄ alkyl, —C₁₋₄ alkyl-CO₂H, and phenyl, wherein the —C₁₋₄ alkyl, —C₁₋₄ alkyl-CO₂H, and phenyl groups are either unsubstituted or substituted with one to three substituents selected from the group consisting of halogen, —OH, —OMe, —NH₂—NO₂₁ unsubstituted benzyl, and benzyl substituted with one to three substituents selected from the group consisting: of halogen, —OH, —OMe, —NH₂, and —NO₂;
X₁ and X₂ are independently selected from the group consisting of O and S; and
X₃ is N or CR_{d} wherein R_{d} is selected from the group consisting of:
   a) hydrogen;
   b) C₁₋₆ alkyl, C₂₋₆ alkenyl, or C₂₋₆ alkynyl; wherein said alkyl, alkenyl, or alkynyl is either unsubstituted or substituted with one or more substituents selected from the group consisting of hydroxy, alkoxy, amino, monoalkylamino, dialkylamino, cycloalkyl, aryl, heterocyclyl, aralkyl, heterocyclylalkyl, acylamino, alkylaminocarbonyl, alkylsulfonylamino, and alkylaminosulfonyl;
   c) substituted or unsubstituted aryl; and
   d) substituted or unsubstituted heterocyclyl.

In some embodiments of this invention, R₁ is C₁₋₆ alkyl. In some embodiments, R₂ is C₁₋₆ alkyl. In some embodiments, R₃ is hydrogen. In some embodiments, R₄ is a single bond.

In some embodiments of the invention, R₅ is a substituted phenyl. In other embodiments, R₅ is a substituted bicyclic or tricyclic group selected from the group consisting of:

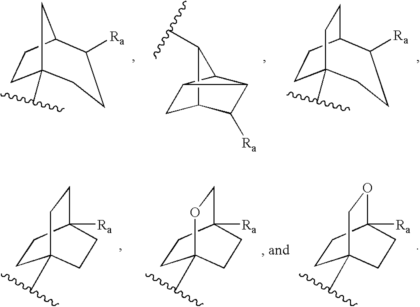

In yet other embodiments, R₅ is 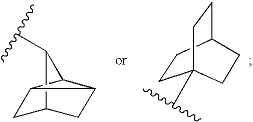
wherein said R₅ is either unsubstituted or substituted with one or more Rₐ groups selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino) (R_{b}) acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phosphono, substituted, or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl (alkyl)amino-, R_{b}-alkyl (alkyl) carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl.

In some embodiments of this invention, Rₐ is selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or -unsubstituted; heterocyclylaminocarbonyl, (amino)(R_{b})acylhydrazinylcarbonyl-, (amino) (R_{b}) acyloxycarboxy-, (hydroxy) (carboalkoxy)alkylcarbamoyl, acyloxy, aldehydo, alkenylsulfonylamino, alkoxy, alkoxycarbonyl, alkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkyl (alkyl)amino-, R_{b}-alkyl(alkyl) carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, cyano, cycloalkylamino, dialkylaminoalkylcarbamoyl, halogen, heterocyclylalkylamino, hydroxy, oximino, phosphate, substituted aralkylamino, substituted heterocyclyl, substituted heterocyclyl-sulfonylamino, sulfoxyacylamino, and thiocarbamoyl.

In other embodiments of this invention Rₐ is selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, R_{b}-, R_{b}-alkoxy-, and substituted or unsubstituted heterocyclyl; and
(b) alkoxycarbonylalkylamino, cyano, and hydroxy.

In some embodiments of the invention, X₁ is O.

In some embodiments, X₂ is O. In some embodiments, X₃ is N.

In some embodiments of the invention, each of R₁ and R₂ is C₂₋₄ alkyl; R₃ is hydrogen; R₄ is a single bond; each of X₁ and X₂ is O; and X₃ is N. In other embodiments of the invention, each of R₁ and R₂ is independently C₂₋₄ alkyl; R₃ is hydrogen; R₄ is a single bond; each of X₁ and X₂ is O; X₃ is N; and R₅ is phenyl substituted with Rₐ.

In other embodiments of the invention, each of R₁ and R₂ is C₂₋₄ alkyl; R₃ is hydrogen; R₄ is a single bond; each of X₁ and X₂ is O; X₃ is N; and R₅ is phenyl substituted with Rₐ; and Rₐ is selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, R_{b}-, and R_{b}-alkoxy-; and
(b) alkoxycarbonylalkylamino, R_{b}-alkoxy-, cyano, substituted or unsubstituted heterocyclyl, and hydroxy.

In yet other embodiments of the invention, each of R₁ and R₂ is C₂₋₄ alkyl; R₃ is hydrogen; R₄ is a single bond; each of X₁ and X₂ is O; X₃ is N; and R₅ is phenyl substituted with Rₐ; and Rₐ is cyano.

In some embodiments of the invention, each of R₁ and R₂ is independently C₂₋₄ alkyl; R₃ is hydrogen; R₄ is a single bond; each of X₁ and X₂ is O; and X₃ is N; and R₅ is 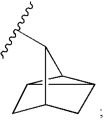
wherein said R₅ is either unsubstituted or substituted with one or more Rₐ groups selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino). (R_{b}) acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylaminoalkylamino, dialkylphosphono, haloalkylsulfonylamino, heterocyclylalkylamino, heterocyclylcarbamoyl, hydroxy, hydroxyalkylsulfonylamino, oximino, phosphono, substituted or unsubstituted aralkylamino, substituted or unsubstituted arylcarboxyalkoxycarbonyl, substituted or unsubstituted: heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl(alkyl)amino-, R_{b}-alkyl(alkyl)carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclyl carbonyl, aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl.

In another embodiment of the invention, each of R₁ and R₂ is C₂₋₄ alkyl; R₃ is hydrogen; R₄ is a single bond; each of X₁ and X₂ is O; and X₃ is N; and R₅ is 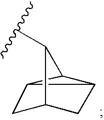
wherein said R₅ is either unsubstituted or substituted with one or more Rₐ groups selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, R_{b}-, and R_{b}-alkoxy-; and
(b) alkoxycarbonylalkylamino, R_{b}-alkoxy-, cyano, substituted or unsubstituted heterocyclyl, and hydroxy.

In another embodiment each of R₁ and R₂ is C₂₋₄ alkyl; R₃ is hydrogen; R₄ is a single bond; each of X, and X₂ is O; and X₃ is N; and R₅ is 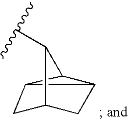
wherein said R₅ is either unsubstituted or substituted with one or more Rₐ groups selected from the group consisting of C₂₋₅ alkyl that is substituted with one or more substituents selected from the group consisting of amino, monoalkylamino, and dialkylamino.

In some embodiments of the invention, each of R₁ and R₂ is C₂₋₄ alkyl; R₃ is hydrogen; R₄ is a single bond; each of X₁ and X₂ is O; X₃ is N; and R₅ is 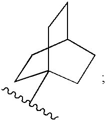
wherein said R₅ is either unsubstituted or substituted with one or more Rₐ groups selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b})acylhydrazinylcarbonyl-, (amino) (R_{b})acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phosphono, substituted or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl(alkyl)amino-, R_{b}-alkyl(alkyl)carbamoyl-, R_{b}-alkylamino-, R_{b}-alkylcarbamoyl-,
R_{b}-alkylsulfonyl-, R_{b}-alkylsulfonylamino, R_{b}-alkylthio, R_{b}-heterocyclylcarbonyl, aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl.

In other embodiments of the invention, each of R₁ and R₂ is C₂₋₄ alkyl; R₃ is hydrogen; R₄ is a single bond; each of X₁ and X₂ is O; X₃ is N; R₅ is 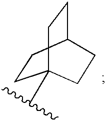
and wherein said R₅ is either unsubstituted or substituted with one or more Rₐ groups selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, R_{b}-, and R_{b}-alkoxy-; and
(b) alkoxycarbonylalkylamino, R_{b}-alkoxy-, cyano, substituted or unsubstituted heterocyclyl, and hydroxy.

In yet another embodiment of the invention, each of R₁ and R₂ is C₂₋₄ alkyl; R₃ is hydrogen; R₄ is a single bond; each of X₁ and X₂ is O; X₃ is N; R₅ is 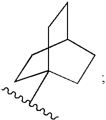
and wherein said R₅ is either unsubstituted or substituted with one or more Rₐ groups selected from the group consisting of:
(a) C₁₋₄ alkyl or C₂₋₄ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, and R_{b}-; and
(b) R_{b}-alkoxy- and substituted heterocyclyl.

In some embodiments of the invention, each of R₁ and R₂ is propyl; R₃ is hydrogen; R₄ is a single bond; R₅ is phenyl substituted with one or more Rₐ groups, 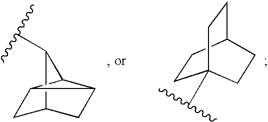
wherein said bicyclic or tricyclic group is either unsubstituted or substituted with one or more Rₐ groups; and
Rₐ is selected from the group consisting of:
   (a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, R_{b}-, R_{b}-alkoxy-, and substituted or unsubstituted heterocyclyl; and
   (b) alkoxycarbonylalkylamino, cyano, and hydroxy; each of X₁ and X₂ is O; and
X₃ is N.

In a preferred embodiment, the compound of formula (I) used in the method of this invention is 3-[4-(2,6-dioxo-1,3-dipropyl-2,3,6,7-tetrahydro-1H-purin-8-yl)-bicyclo[2.2.2]oct-1-yl]-propionic acid.

In some embodiments, the A_{2b} adenosine receptor antagonist is administered to a human.

In some embodiments, the A_{2b} adenosine receptor antagonist used in the method of this invention is formulated together with a pharmaceutically suitable carrier into a pharmaceutically acceptable composition.

The invention is useful in the treatment of patients having undergone an ischemic event or in which an ischemic event is imminent. Examples of ischemic events include acute coronary syndrome (including myocardial infarction), stroke, organ transplantation, kidney ischemia, shock, and organ transplantation surgery.

In some embodiments, the method of this invention includes administering the A_{2b} adenosine receptor antagonist within two days before or after the ischemic event. In another embodiment, the method includes administering the A_{2b} adenosine receptor antagonist within two days after the ischemic event.

In some embodiments, the compound used in the methods of the invention exhibits an affinity for an A_{2b} adenosine receptor that is at least 10-fold greater than the affinity for an A₂ₐ adenosine receptor or an A₃ adenosine receptor. In other embodiments, the compound used in the methods of the invention further exhibits an affinity for an A₁ adenosine receptor that is at least 10-fold greater than the affinity for an A₂ₐ adenosine receptor or an A₃ adenosine receptor.

In some embodiments, the compound used in the methods of the invention exhibits a Kᵢ value for an A_{2b} adenosine receptor which is below 500 nM. In other embodiments, the compound used in the method of the invention exhibits a Kᵢ value for an A_{2b} adenosine receptor which is below 200 nM.

In some embodiments, the invention relates to a method of treating a disease or disorder mediated by activation of an A_{2b} adenosine receptor comprising administering to a mammal in need thereof an effective amount of a compound of formula (I)as described above.

In some embodiments, the invention relates to a method of limiting tissue necrosis resulting from an ischemic event, in a mammal that has undergone an ischemic event, or in which an ischemic event is imminent using an A_{2b}, adenosine receptor antagonist.

In some embodiments, the invention relates to a method of limiting infarction size following myocardial infarction, in a mammal that has undergone myocardial infarction, or in which myocardial infarction is imminent using an A_{2b} adenosine receptor.

### BRIEF DESCRIPTION OF THE DRAWINGS

FIG. 1 depicts myocardial infarct size data from protocol I (see Example 2). Panel A depicts the risk region size in the four experimental groups expressed as a percentage of the left ventricle. Panel B depicts the infarct size as a percentage of the risk region., Panel C depicts the infarct size expressed as a percentage of the left ventricle. Panel D reflects a plot of infarct size expressed as a percentage of the risk region and transmural collateral blood flow measured 30 minutes after coronary occlusion.

FIG. 2 depicts myocardial infarct size data from protocol II (See Example 3). Panel A depicts the risk region size in the four experimental groups expressed as a percentage of the left ventricle. For the purposes of comparison, the control group from protocol I was also included. Panel B depicts the infarct size as a percentage of the risk region. Panel C depicts the infarct size expressed as a percentage of the left ventricle. Panel D reflects a plot of infarct size expressed as a percentage of the risk region and transmural collateral blood flow measured 30 minutes after coronary occlusion.

FIG. 3 depicts myocardial infarct size data from protocol III (see Example 4). Panel A depicts the risk region size in the four experimental groups expressed as a percentage of the left ventricle. Panel B depicts the infarct size as a percentage of the risk region. Panel C depicts the infarct size expressed as a percentage of the left ventricle. Panel D reflects a plot of infarct size expressed as a percentage of the risk region and transmural collateral blood flow measured. 30 minutes after coronary occlusion.

FIG. 4 depicts competitive binding of BG9928 on recombinant human A₁ adenosine receptors. Membranes (50 μg membrane protein) made from HEK 293 cells stably expressing human A₁ adenosine receptors, 0.92 nM radioligand [³H]-DPCPX, and varying concentrations of BG9928 were incubated in triplicate in 0.1 ml buffer HE plus 2 units/mL adenosine deaminase for 2.5 hours at 21° C. Nonspecific binding was measured in the presence of 10 μM NECA. Binding assays were terminated by filtration. (N=1).

FIG. 5 depicts competitive binding of BG9928 on recombinant human A₂ₐ adenosine receptors. Membranes (50 μg membrane portein) made from HEK 293 cells stably expressing human A₂ₐ adenosine receptors, 1.16 nM radioligand [³H]-ZM241385 and varying concentrations of BG9928 were incubated in triplicate in 0.1 ml buffer HE plus 2 units/mL adenosine deaminase-for 2.5 hours at 21° C. Nonspecific binding was measured in the presence of 10 μM XAC. Binding assays were terminated by filtration. (N=1).

FIG. 6 depicts competitive binding of BG9928 on recombinant human A_{2b} adenosine receptors. Membranes (40-70 μg membrane protein) made from HEK 293 cells stably expressing recombinant human A_{2b} adenosine receptors, 30-40 nM radioligand [³H]-ZM241385, and varying concentrations of BG9928 were incubated in triplicate in 0.1 ml buffer HE plus 2 units/mL adenosine deaminase for 2.5 hours at 21° C. Nonspecific binding was measured in the presence of 10 μM NECA. Binding assays were terminated by filtration. (N=3).

FIG. 7 depicts one point binding of BG9928 on recombinant human A₃ adenosine receptors. Membranes made from HEK 293 cells stably expressing recombinant human A₃ adenosine receptors (50 μg membrane protein) and 0.12 nM radioligand [¹²⁵I]-AB-MECA either alone, with 10 μM IB-MECA or with 10 μM BG9928 were incubated in triplicate in 0.1 ml buffer HE plus 2 units/mL adenosine deaminase for 2.5 hours at 21° C. Binding assays were terminated by filtration. (N=2).

FIG. 8 depicts FLIPR assay of BG9928 with recombinant human A₁ adenosine receptors stably expressed in CHO-K1 cells. FLIPR assays measuring the response of CHO-K1 cells expressing recombinant human A₁ adenosine receptors to increasing concentrations of agonist (CPA) (top graph), and to determine the IC₅₀ (concentration at which a 50% of response was obtained and then K_{B} values for the antagonist BG9928 at a fixed agonist concentration (200 nM CPA) using the null method (bottom graph).

FIG. 9 depicts FLIPR assay of BG9928 with recombinant human A_{2b} adenosine receptors stably expressed in HEK-293 cells. FLIPR assays measuring the response of HEK-293 cells stably expressing recombinant human A_{2b} adenosine receptors to increasing concentrations of the agonist (NECA) (top graph), and to determine IC₅₀ (the concentration at which a 50% response was obtained) and then K_{B} values for the antagonist BG9928 at a fixed agonist concentration (5 μM NECA) using null method (bottom graph).

FIG. 10 depicts FLIPR assay of BG9928 with recombinant human A_{2b} adenosine receptors stably expressed in HEK-293 cells. FLIPR assays measuring the fraction of control response observed with 10, 100, and 300 nM BG9928 in HEK-293 cells expressing rat A_{2b} adenosine receptors in the presence of increasing concentrations of the agonist (NECA) (top graph). The bottom graph is a Schild analysis of the data presented in the top graph.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable materials and methods are described below. All publications, patent applications, patents, and other references mentioned herein are incorporated by reference in their entirety. In addition, the materials, methods, and examples are illustrative only and are not intended to be limiting.

Throughout the specification, the word “comprise” or variations such as “comprises” or “comprising” will be understood to imply the inclusion of a stated integer or groups of integer but not the exclusion of any other integers or groups of integers.

As used herein, an “alkyl” group is a saturated aliphatic hydrocarbon group. An alkyl group can be straight or branched, and can have, for example, from 1 to 6 carbon atoms in a chain. Examples of straight chain alkyl groups include, but are not limited to ethyl and butyl. Examples of branched alkyl groups include, but are not limited to, isopropyl and t-butyl. An alkyl group may be optionally substituted with one or more substituents such as alkoxy, amino, nitro, carboxy, carboalkoxy, cyano, halo, hydroxy, mercaptyl, trihalomethyl, sulfoxy, or carbamoyl.

As used herein, an “alkenyl” group is an aliphatic carbon group that has at least one double bond. An alkenyl group can be straight or branched, and can have, for example, from 3 to 6 carbon atoms in a chain and 1 or 2 double bonds. Examples of alkenyl groups include, but are not limited to, allyl and isoprenyl. An alkenyl group may be optionally substituted with one or more substituents such as alkoxy, amino, nitro, carboxy, carboalkoxy, cyano, halo, hydroxy, mercaptyl, trihalomethyl, sulfoxy, or carbamoyl.

As used herein, an “alkynyl” group is an aliphatic carbon group that has at least one triple bond. An alkynyl group can be straight or branched, and can have, for example, from 3 to 6 carbon atoms in a chain and 1 to 2 triple bonds. Examples of alkynyl groups include, but are not limited to, propargyl and butynyl. An alkynyl group may be optionally substituted with one or more substituents such as alkoxy, amino, nitro, carboxy, carboalkoxy, cyano, halo, hydroxy, mercaptyl, trihalomethyl, sulfoxy, or carbamoyl.

As used herein, an “aryl” group is a phenyl or naphthyl group, or a derivative thereof. A “substituted aryl” group is an aryl group that is substituted with one or more substituents such as alkyl, alkoxy, amino, nitro, carboxy, carboalkoxy, cyano, alkylamiino, dialkylamino, halo, hydroxy, hydroxyalkyl, mercaptyl, alkylmercaptyl, trihaloalkyl, carboxyalkyl, sulfoxy, or carbamoyl.

As used herein, an “aralkyl” group is an alkyl group that is substituted with an aryl group. An example of an aralkyl group is benzyl.

As used herein, an “cycloalkyl” group is an aliphatic ring of, for example, 3 to 8 carbon atoms. Examples of cycloalkyl groups include cyclopropyl and cyclohexyl.

As used herein, an “acyl” group is a straight or branched alkyl-C(═O)— group or a formyl group. Examples of acyl groups include alkanoyl groups (e.g., having from 1 to 6 carbon atoms in the alkyl group). Acetyl and pivaloyl are examples of acyl groups. Acyl groups may be substituted or unsubstituted.

As used herein, a “carbamoyl” group is a group having the structure H₂N—CO₂—. “Alkylcarbamoyl” and “dialkylcarbamoyl” refer to carbamoyl groups in which the nitrogen has one or two alkyl groups attached in place of the hydrogens, respectively. By analogy, “arylcarbamoyl” and “arylalkylcarbamoyl” groups include an aryl group in place of one of the hydrogens and, in the latter case, an alkyl group in place of the second hydrogen.

As used herein, a “carboxyl” group is a —COOH group.

As used herein, an “alkoxy” group is an alkyl-O— group in which “alkyl” is as previously described.

As used herein, an “alkoxyalkyl” group is an alkyl group as previously described, with a hydrogen replaced by an alkoxy group, as previously described.

As used herein, a “halogen” or “halo” group is fluorine, chlorine, bromine or iodine.

As used herein, a “heterocyclyl” group is a 5 to about 10 membered ring structure, in which one or more of the atoms in the ring is an element other than carbon, e.g., N, O, S. A heterocyclyl group can be aromatic or non-aromatic, i.e., can be saturated, or can be partially or fully unsaturated. An aromatic heterocyclyl group may also be referred to as a “heteroaryl” group. Examples of heterocyclyl groups include pyridyl, imidazolyl, furanyl, thienyl, thiazolyl, tetrahydrofuranyl, tetrahydropyranyl, morpholinyl, thiomorpholinyl, indolyl, indolinyl, isoindolinyl, piperidinyl, pyrimidinyl, piperazinyl, isoxazolyl, isoxazolidinyl, tetrazolyl, and benzimidazolyl.

As used herein, a “substituted heterocyclyl” group is a heterocyclyl group wherein one or more hydrogens are replaced-by substituents such as alkoxy, alkylamino, dialkylamino, carbalkoxy, carbamoyl, carboxyl, cyano, halo, trihalomethyl, hydroxy, carbonyl, thiocarbonyl, hydroxyalkyl or nitro.

As used herein, a “hydroxyalkyl” means an alkyl group substituted by a hydroxy group.

As used herein, a “sulfamoyl” group has the structure —S(O)₂NH₂. “Alkylsulfamoyl” and “dialkylsulfamoyl” refer to sulfamoyl groups in which the nitrogen has one or two alkyl groups attached in place of the hydrogens, respectively. By analogy, “arylsulfamoyl” and “arylalkylsulfamoyl” groups include an aryl group in place of one of the hydrogens and, in the latter case, an alkyl group in place of the second hydrogen.

As used herein, an “antagonist” is a molecule that binds to a receptor without activating the receptor. It competes with the endogenous ligand for this binding site and, thus, reduces the ability of the endogenous ligand to stimulate the receptor.

As used herein, a “selective antagonist” is an antagonist that binds to a specific subtype of adenosine receptor with higher affinity than to other subtypes. An “A_{2b} selective antagonist” as used herein is an antagonist having high affinity for A_{2b} receptors and has (a) nanomolar binding affinity for the A_{2b} receptor subtype and (b) at least 10 times, more preferably 50 times, and most preferably 100 times, greater affinity for the A_{2b} subtype than for A₂ₐ and A₃ receptor subtypes. The A_{2b} selective antagonist may optionally have affinity for the A₁ receptor subtype and have (a) nanomolar binding affinity for the A₁ receptor subtype and (b) at least 10 times, more preferably 50 times, and most preferably 100 times, greater affinity for the A₁ subtype than for A₂ₐ and A₃ receptor subtypes.

As used herein, “infarction” means localized necrosis resulting from obstruction of the blood supply to a tissue (e.g., mycardium).

As used herein, “ischemia” means an inadequate blood supply (circulation) to a local area (i.e., organ or tissue) due to blockage of the blood vessels to the area. Ischemia includes complete cessation of blood flow and oxygen delivery to a tissue as well as hypoxia whereby there is a substantial reduction in oxygen delivery to a tissue.

As used herein “reperfusion” means the restoration of blood flow to an organ or tissue.

As used herein, “ischemia reperfusion injury” refers to the injury to a tissue caused by ischemia followed by reperfusion.

As used herein, “pharmaceutically acceptable” means an amount effective in treating or preventing a condition characterized by an elevated adenosine concentration and/or increased sensitivity to adenosine.

As used here, the term “patient” means an animal, including a mammal (e.g., a human).

As used herein, “pharmaceutically acceptable carrier or adjuvant” means a non-toxic carrier or adjuvant that may be administered to an animal, together with a compound of this invention, and which does not destroy the pharmacological activity thereof.

Pharmaceutically acceptable anion salts include salts of the following acids methanesulfonic, hydrochloric, hydrobromic, sulfuric, phosphoric nitric, benzoic, citric, tartaric, fumaric, maleic, CH₃—(CH₂)ₙ COOH where n is 0-4, HOOC—(CH₂)ₙ—COOH where n is as defined above.

When solvent pairs are used, the ratios of solvents used are volume/volume (v/v).

When the solubility of a solid in a solvent is used the ratio of the solid to the solvent is weight/volume (wt/v).

In addition, the following abbreviations will apply throughout the specification:
BCA refers to Bicinchoninic acid.,
BG9928 refers to 3-[4-(2,6-dioxo-1,3-dipropyl-2,3,6,7-tetrahydro-1H-purin-8-yl)-bicyclo[2.2.2]oct-1-yl]-propionic acid.
(Ca²⁺)i refers to intracellular calcium.
CCD refers to Charged Coupled Device.
CPA refers to N6-cyclopentyladenosine.
CPM refers to counts per minute.
DPM refers to disintegrations per minute.
DR refers to the concentration ratio, i.e., concentration of agonist producing a defined response (usually, but not necessarily, 50% of maximum) in the presence of an antagonist, divided by the concentration producing the same response in the absence of antagonist.
EDTA refers to ethylenediaminetetraacetic acid.
FLIPR refers to Fluorescence Imaging Plate Reader.
[³H]-BG9928 refers to tritium-labeled BG9928.
[³H]-DPCPX refers to tritium labeled 8-cyclopentyl-1,3-dipropylxanthine, a competitive substrate for A₁ and A_{2b} adenosine receptors.
[³H]-ZM241385 refers to tritium labeled 4-(2-[7-amino-2-(furyl)(1,2,4)triazolo(2,3-a)(1,3,5)triazin-5-ylaminoethyl)phenol, a competitive substrate for A₂ₐ adenosine receptors.
[I] refers to the concentration of the free radioligand.
[¹²⁵I]AB-MECA refers to [¹²⁵Iodine]-labeled N6-(4-aminobenzyl)-9-(5-(methylcarbonyl)-β-D-ribofuranosyl) adenine.
IB-MECA refers to 1-Deoxy-1-[6-[[(3-iodophenyl)methyl]amino]-9H-purin-9-yl]-N-methyl-βN6-(4-aminobenzyl)-9-(5-(methylcarbonyl)-β-D-ribofuranuronamide.
IC₅₀ refers to the concentration of agent which inhibits 50% of activity being measured.
K_{B} refers to antagonist dissociation constant.
K_{D} refers to the dissociation constant for a radiolabeled drug determined by saturation analysis.
K_{I} refers to the inhibition constant for a drug; the concentration of competing ligand in a competition assay that would occupy 50% of the receptors if no radioligand were present.
AB-MECA refers to N6-(4-aminobenzyl)-9-(5-(methylcarbonyl)-β-D-ribofuranosyl)adenine.
N refers to number of observations.
NECA refers to 5′N-ethylcarboxamidoadenosine.
pA₂ refers to a logarithmic measure of the potency of an antagonist; the negative log of the concentration of antagonist that would produce a 2-fold shift in the concentration-response curve for an agonist.
PMSF refers to phenylmethyl sulphonyl fluoride. RFU refers to Relative Fluorescence Units.
³H-R-PIA refers to [³H]-R-N⁶-phenylisopropyladenosine (radioligand for A3 adenosine receptors).
Schild plot refers to a graph of log (concentration ratio −1), i.e., log (DR-1), against log (antagonist concentration). The intercept on the log concentration axis is equal to the pA₂ value, while the slope gives information about the nature of antagonism.
SD refers to standard deviation.
SEM refers to the standard error of the mean
XAC refers to xanthine amino congener.

In general, the invention features highly potent and selective antagonists of the A_{2b} adenosine receptor. In some embodiments, the compounds of the invention may optionally be selective antagonists of the A₁ adenosine receptor.

Synthesis of the Adenosine Antagonist Compounds

Compounds useful in the invention may be prepared by conventional methods known in the art. For example, the synthesis of the compounds of formula I is described in International Publication Nos. WO01/34604 and WO01/34610.

Two general methods are described herein. Each of them employs a common starting material, 1,3-disubstituted-5,6-diaminouracil (compound (VI)), as shown in the two schemes below. 1,3-Disubstituted-5,6-diaminouracils can be prepared by treating the corresponding symmetrically or unsymmetrically substituted urea with cyanoacetic acid, followed by nitrosation and reduction (see, e.g., J. Org. Chem. 16, 1879, 1951; Can J. Chem. 46, 3413, 1968, incorporated herein by reference). Unsymmetrically substituted xanthines can be accessed via the method of Mueller (J. Med. Chem. 36, 3341, 1993, incorporated herein by reference). In this method, 6-aminouracil is monoalkylated specifically at N3 of the uracil under Vorbruggen conditions. Alternatively, unsubstituted N1 or N3 position can be functionalized (e.g., alkylation) in the last stage of synthesis.

In the first general method, a 1,3-disubstituted-5,6-diaminouracil (compound (VI)) can first undergo a ring closure reaction to produce a xanthine intermediate that is unsubstituted at the 8-position. This intermediate, in turn, can couple with a precursor compound of the Z-R₃ moiety to produce the desired 8-substituted xanthines. Referring to scheme 1 below, the starting material 1,3-disubstituted-5,6-diaminouracil (i.e., compound (VI)) first-reacts with HC(OEt)₃ to undergo a ring closure reaction to produce a xanthine intermediate that is unsubstituted at the 8-position (i.e., compound (A)). This intermediate, after being protected by an amino protecting group (e.g., with THP or BOM at the N7 position), further undergoes a coupling reaction, in the presence of a strong base (e.g., n-butyl-lithium (nBuLi) or lithium di-isopropyl-amide (LDA)), with a precursor compound of the Z-R₃ moiety (e.g., an aldehyde or a ketone) to produce an alcohol (i.e., compound (C)). The hydroxyl group of the alcohol can then be reacted to convert the alcohol to an amine, a mercaptan, an ether, a lactone (e.g., compound (E)), or other functionalized compound, by methods well known to those of ordinary skill in the art. The N7 protection can then be removed to obtain a deprotected product (i.e., compound (F)), which can be further functionalized to yield compounds of this invention.

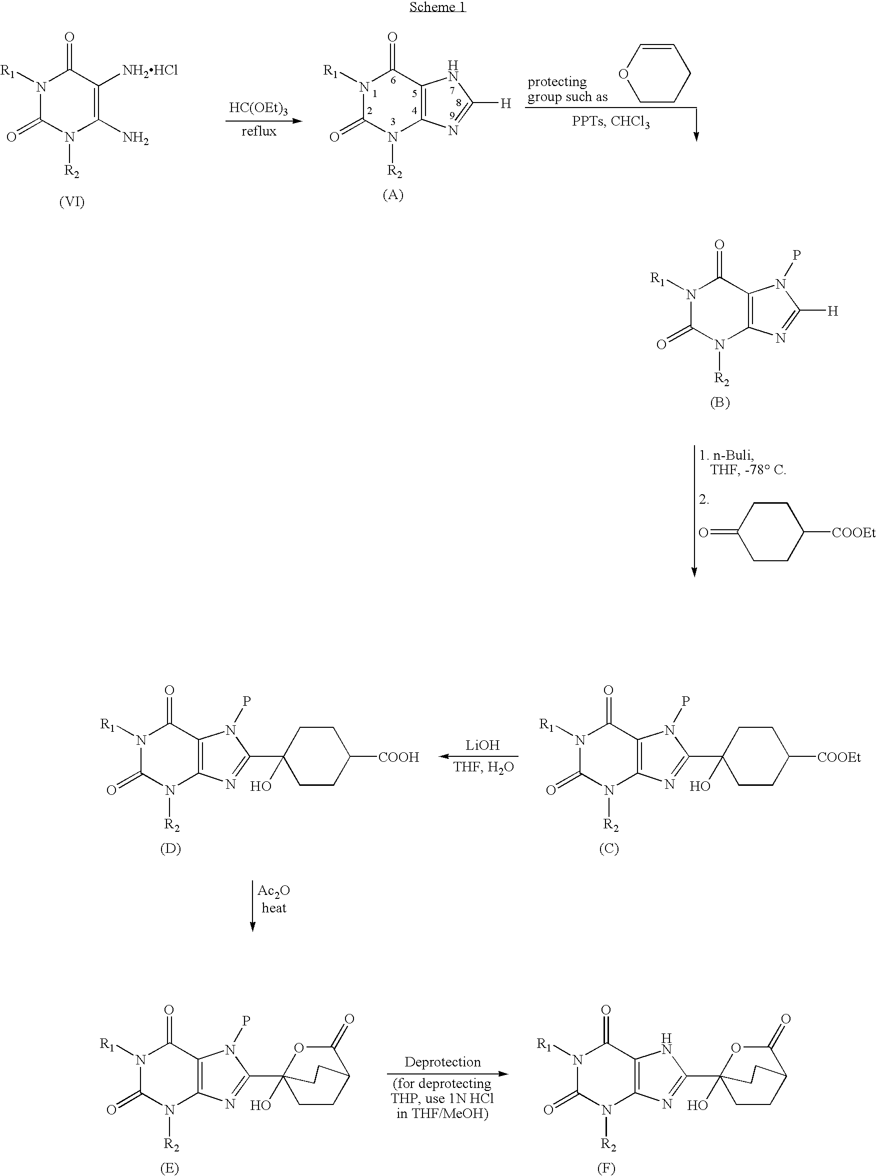

In the second general method., compounds of the invention can be prepared by reacting the starting material, a 1,3-disubstituted-5,6-diaminouracil, with a precursor compound of the Z-R₃ moiety (e.g., aldehydes or carboxylic acids or carboxylic acid chlorides) to form a 6-amide substituted uracil intermediate, which in turn, can undergo a ring closure reaction to yield to a desired xanthine compound. Referring to scheme 2 below, the starting material 1,13-disubstituted-5,6-diaminouracil (i.e., compound (VI)) first couples with a di-carboxyl/ester-substituted precursor compound of the Z-R₃ moiety, HOOC-Z-R₃—COORₐ (i.e., compound (G); Rₐ represents H, C₁₋₅ alkyl, or benzyl, the phenyl ring being optionally substituted with 1-3 substituents selected from the group consisting of halo, hydroxyl, or C₁₋₃ alkoxy) to yield a 6-amide substituted uracil intermediate (i.e., compound (H)) by reactions which are well known to one of ordinary skill in the art (e.g., by employing coupling reagents such as benzotriazol-1-yloxytris(dimethylamino)-phosphonium hexafluorophosphate (BOP), C-benzo-triazol-1-yl-N,N,N′,N′-tetramethyluronium hexafluorophosphate (HBTU), or O-(7-azabenzottiazol-1-yl)-N,N,N′,N′-tetramethyluronium hexafluorophosphate (HATU)). Examples of compound (G) include bicyclo[3.2.1]octane-1,5-dicarboxylic acid monomethyl ester and bicyclo[2.2.2]octane-1,4-dicarboxylic acid monoethyl ester. The uracil intermediate can then undergo a ring closure reaction in a basic condition (e.g., by employing KOH and isopropyl alcohol) to yield a xanthine compound (i.e., compound (J)), which can undergo further functionalization to produce various compounds of the invention.

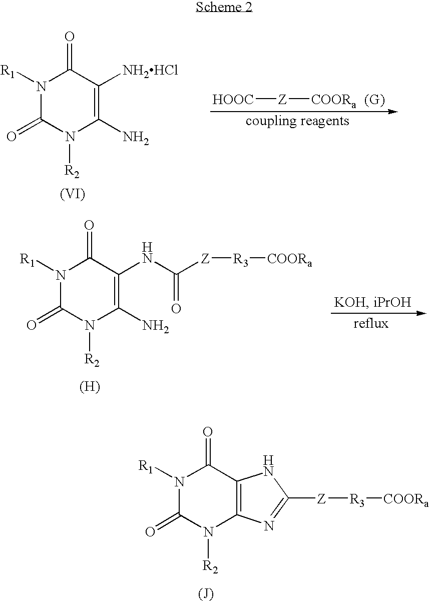

The desired aldehydes, ketones, carboxylic acids and carboxylic acid chlorides are commercially available (e.g., from Aldrich Chemical Co., Inc., Milwaukee, Wis.) or can be readily prepared from commercially available materials by well-known synthetic methods. Such synthetic methods include, but are not limited to, oxidation, reduction, hydrolysis, alkylation and Wittig homologation reactions. For references regarding the preparation of bicycloalkane carboxylic acids of the invention (e.g., compound (III), which is an example of compound (G)), see, e.g., Aust. J. Chem. 38, 1705, 1985; Aust J. Chem. 0.39, 2061, 1986; J. Am. Chem. Soc. 75,637, 1953; J. Am. Chem. Soc. 86, 5183, 1964; J. Am, Chem. Soc. 102, 6862, 1980; J. Org. Chem. 46, 4795, 1981; and J. Org. Chem. 60, 6873, 1995.

There are many methods to further functionalize compound (J), which contains a carboxylic acid or ester attached to the R₃ moiety. For example, compound. (J) can be converted to the corresponding acrylic acid derivative. One way is to first hydrolyze the ester group of compound (J) (provided that Rₐ is not H) to give the corresponding carboxylic, acid, reduce the carboxylic acid to the corresponding alcohol, oxidize the alcohol to the corresponding aldehyde, and then perform a Wadsworth-Horner-Emmons or Witting reaction to form the corresponding acrylic acid derivative. Compound (J) can also be transformed directly to its corresponding alcohol. A different variation is to transform compound (J) directly to its corresponding aldehyde. A further variation, is to transform an ester-containing compound (J) to its corresponding carboxylic acid, and then directly to the aldehyde. Alternatively, one can functionalize the precursor compound of the Z-R₃ moiety before coupling to the or 1,3-disubstituted-8-unsubstituted xanthine in scheme 1 or the 1,3-disubstituted-5,6-diaminouracil in scheme 2. Further, compounds of this invention can be prepared on solid support (e.g., Wang resin).

The synthesis of 3-[4-(2,6-dioxo-1,3-dipropyl-2,3,6,7-tetrahydro-1H-purin-8-yl)-bicyclo[2.2.2]oct-1-yl]-propionic acid (BG9928) is described in International publication WO01/34610.

In some embodiments, the compounds may be in the form of an achiral compound, an optically active compound, a pure diastereomer, a mixture of diastereomers, a prodrug or a pharmacologically acceptable salt thereof.

In some embodiments, of the invention, the compounds of formula I exhibit an affinity for the A_{2b} adenosine receptor that is at least 10-fold greater than, the affinity for the A₂ₐ adenosine receptor or the A₃ adenosine receptor. In other embodiments, the compounds of formula I exhibit an affinity for the A_{2b} adenosine receptor that-is at least 50-fold greater than the affinity for the A₂ₐ adenosine receptor or the A₃ adenosine receptor. In yet other embodiments, the compounds of formula I exhibit an affinity for the A_{2b} adenosine receptor that is at least 100-fold greater than the affinity for the A₂ₐ adenosine receptor or the A₃ adenosine receptor. In some embodiments, in addition to the affinity for the A_{2b} adenosine receptor, the compounds of formula I optionally exhibit an affinity for the A₁ adenosine receptor.

In some embodiments of the invention, the compounds of formula I exhibit a Ki value for the A_{2b} adenosine receptor which is below 500 nM. In other embodiments of the invention, the compounds of formula I exhibit a Ki value for the A_{2b} adenosine receptor which is below 200 nM. In yet other embodiments of the invention, the compounds of formula I exhibit a Ki value for the A_{2b} adenosine receptor which is below 10 nM.

Production of A_{2b} adenosine Receptor Antibodies

The invention also encompasses the use of antibodies raised against the A_{2b} adenosine receptor, as antagonists of the receptor. Such antibodies block the ligand (e.g., adenosine) binding site on the A_{2b} adenosine receptor or prevent the ligand (e.g., adenosine) from binding to the receptor.

The A_{2b} adenosine receptor may be used to elicit polyclonal or monoclonal antibodies which bind to the A_{2b} adenosine receptor using a variety of techniques well known to those of skill in the art. Alternatively, peptides corresponding to specific regions of the A_{2b} adenosine receptor may be synthesized and used to create immunological reagents according to well known methods.

The human A_{2b} adenosine receptor has been cloned and the DNA sequence encoding the receptor as well as the protein sequence for the receptor have been identified (Rivkee et al., Mol. Endocrinol., 6, pp. 1598-1604 (1992); Pierce et al., Biochem. Biophys. Res. Commun., 187, pp. 86-93 (1992); Reppert et al., U.S. Pat. No. 5,516,894).

Antibodies directed against the A_{2b} adenosine receptor of this invention are immunoglobulin molecules or portions thereof that are immunologically reactive with the A_{2b} adenosine receptor of the present invention. More preferably, the antibodies used in the methods of the invention are immunologically reactive with the ligand binding domain of the A_{2b} adenosine receptor.

Antibodies directed against the A_{2b} adenosine receptor may be generated by immunization of a suitable host. Such antibodies may be polyclonal or monoclonal. Preferably they are monoclonal. Production of polyclonal and monoclonal antibodies is within ordinary skill in the art. For a review of methods useful in practicing the invention, see, e.g., Harlow and Lane (1988), Antibodies, A Laboratory Manual, Yelton, D. E. et al. (1981); Ann. Rev. of Biochem., 50, pp. 657-80, and Ausubel et al. (1989); Current Protocols in Molecular Biology (New York: John Wiley & Sons), updated annually. Determination of immunoreactivity with an A_{2b} adenosine receptor may be made by any of several methods well known in the art, including, e.g., immunoblot assay and ELISA.

Monoclonal antibodies with affinities of 108 M or preferably 10⁻⁹ to 10⁻¹⁰ M⁻¹ or stronger are typically made by standard procedures as described, e.g., in Harlow and Lane, (1988) supra. Briefly, appropriate animals are selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to the antigenic A_{2b} adenosine receptor, or alternatively, to selection of libraries of antibodies in phage or similar vectors. See Huse et al., Science, 246, pp. 1275-81 (1989). Antibodies useful in the present invention may be employed with or without modification. Antigens (in this case the A_{2b} adenosine receptor) and antibodies can be labeled by joining, either covalently or non-covalently, a substance which provides for a detectable signal. Various labels and conjugation techniques are known in the art and can be employed in practicing the invention. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins; may be produced (see U.S. Pat. No. 4,816,567).

An antibody of this invention may also be a hybrid molecule formed from immunoglobulin sequences from different species (e.g., mouse and human) or from portions of immunoglobulin light and heavy chain sequences from the same species. An antibody may be a single-chain antibody or a humanized antibody. It may be a molecule that has multiple binding specificities, such as a bifunctional antibody prepared by any one of a number of techniques known to those of skill in the art including the production of hybrid hybridomas, disulfide exchange, chemical cross-linking, addition of peptide linkers between two monoclonal antibodies, the introduction of two sets of immunoglobulin heavy and light chains into a particular cell line, and so forth.

The antibodies of this invention may also be human monoclonal antibodies, for example those produced by immortalized human cells, by SCID-hu mice or other non-human animals capable of producing “human” antibodies, or by the expression of cloned human immunoglobulin genes. The preparation of humanized antibodies is taught by U.S. Pat. Nos. 5,777,085 and 5,789,554.

In sum, one of skill in the-art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

Uses for A_{2b} adenosine Receptor Antagonists

The methods and compositions of this invention may be used to prevent, limit or treat patients having undergone an ischemic eventor in which an ischemic event is imminent. The ischemic event can be, for example, acute coronary syndrome (including myocardial infarction), stroke, organ transplantation, kidney ischemia, shock, and organ transplantation surgery. In some embodiment, the ischemic event is a myocardial infarction.

In some embodiments of the present invention, the A_{2b} adenosine receptor antagonist is administered within ten days before or after the ischemic event. In other embodiments of the present invention, the A_{2b} adenosine receptor antagonist is administered within five days before or after the ischemic event. In yet other embodiments of the present invention, the A_{2b} adenosine receptor antagonist is administered within two days before or after the ischemic event. In other embodiments, the A_{2b} adenosine receptor antagonist is administered within two days after the ischemic event.

The present invention also provides a method of treating a disease or disorder mediated by activation of the A_{2b} adenosine receptor by administering to a mammal in need thereof an pharmaceutically effective or a prophylactically effective amount of an A_{2b} adenosine receptor antagonist of this invention.

The ischemic event often results in necrosis of the tissue affected. The present invention also provides a method of limiting tissue necrosis resulting from an ischemic event comprising identifying a mammal that has undergone an ischemic event or in which an ischemic event is imminent and administering a therapeutically effective or prophylactically effective amount of an A_{2b} adenosine receptor antagonist of this invention. In some embodiments, the A_{2b} adenosine receptor antagonist is administered within ten days before or after the ischemic event. In other embodiments, the A_{2b} adenosine receptor antagonist is administered within five days before or after the ischemic event. In other embodiments, the A_{2b} adenosine receptor antagonist is administered within two days before or after the ischemic event.

Myocardial infarction is the development of myocardial necrosis caused by an imbalance between the oxygen supply and demand of the myocardium and results in myocardial necrosis. Myocardial infarctions are often caused by the rupture of plaque with thrombus formation in a coronary vessel, resulting in an acute reduction of blood supply to a portion of the myocardium. This may result in partial or complete occlusion of the vessel and subsequent myocardial ischemia. Complete occlusion of the coronary vessel for several hours (e.g., 4-6 hours) results in irreversible myocardial necrosis. However, reperfusion within this period can salvage the myocardium and reduce morbidity and mortality. Therefore, the invention also provides a method of limiting the size of an infarction, following a myocardial infarction by identifying a mammal that has undergone a myocardial infarction or in which a myocardial infarction is imminent and administering a therapeutically effective or prophylactically effective amount of an A_{2b} adenosine receptor antagonist of this invention. In some embodiments, the A_{2b} adenosine receptor antagonist of this invention is administered within ten days before or after the ischemic event. In other embodiments, the A_{2b} adenosine receptor antagonist is administered within five days before or after the ischemic event. In other embodiments, the A_{2b} adenosine receptor antagonist is administered within two days before or after the ischemic event.

Pharmaceutical Compositions

The adenosine A_{2b} receptor antagonists may be formulated into pharmaceutical compositions for administration to animals, including humans. These pharmaceutical compositions, preferably include an amount of A_{2b} adenosine receptor antagonist effective to treat, limit or prevent ischemia reperfusion injury and a pharmaceutically acceptable carrier.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered parenterally, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as absolution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis.

The pharmaceutical compositions of this invention may be orally administered in any orally acceptable dosage form including, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of A_{2b} adenosine receptor antagonist that may be combined with the carrier materials to produce a single dosage form will vary depending upon the host treated and the particular mode of administration. The compositions can be formulated so that a dosage of between 0.01-100 mg/kg body weight of the A_{2b} adenosine receptor antagonist is administered to a patient receiving these compositions. In some ebodiments of the invention, the dosage is 0.1-10 mg/kg body weight. The composition may be administered as a single dose, multiple doses or over an established period of time in an infusion.

A specific dosage and treatment regimen for any particular patient will depend upon a variety of factors, including the particular A_{2b} adenosine receptor antagonist, the patient's age, body weight, general health, sex, and diet, and the time of administration, rate of excretion, drug combination, and the severity of the particular disease being treated. Judgment of such factors by medical caregivers is within ordinary skill in the art. The amount of antagonist will also depend on the individual patient to be treated, the route of administration, the type of formulation, the characteristics of the compound used, the severity of the disease, and the desired effect. The amounts of antagonists can be determined by pharmacological and pharmacokinetic principles well-known in the art.

According to some embodiments, the invention provides a method for preventing, limiting or treating ischemia reperfusion injury comprising the step of administering to a patient one of the above-described pharmaceutical compositions.

In order that the invention described herein may be more fully understood, the following examples are set forth. It should be understood that these examples are for illustrative purposes only and are not to be construed as limiting this invention in any manner.

### EXAMPLES

1. Animal Model and General Procedures

The studies were performed in open-chest, barbital-anesthetized dogs instrumented to measure heart rate, blood pressure, left ventricular pressure, and regional myocardial blood flow (radioactive microspheres). A mechanical occluder was placed around a proximal portion of the left anterior descending coronary artery to produce ischemia and reperfusion. At the end of the experiments, infarct size was determined-by histochemical staining (patent blue dye and triphenyltetrazolium) and expressed as a percentage of the region at risk or as a percentage of the entire left ventricle.

2. Pretreatment Experimental Protocol

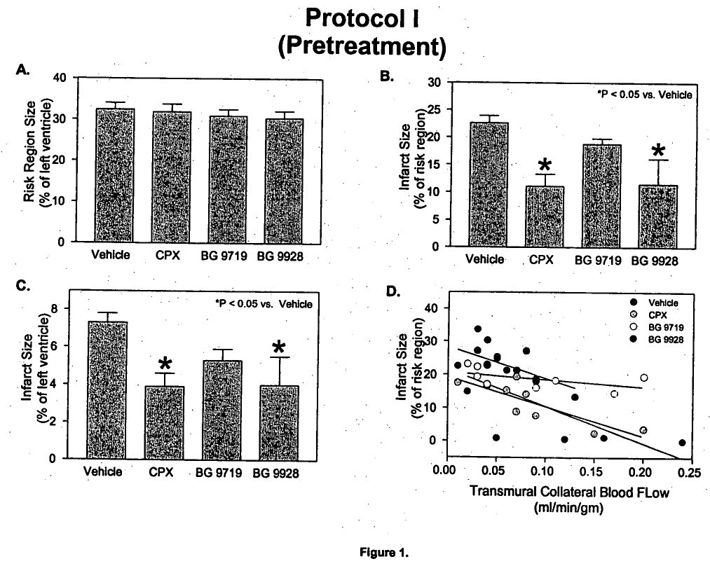

In the pretreatment protocol (see FIG. 1, protocol I), the dogs were subjected to 60 minutes of coronary artery occlusion and 3 hours or reperfusion after which the hearts were removed and infarct size was assessed. Four groups of dogs were randomly assigned to receive vehicle, CPX (8-Cyclopentyl-1,3-dipropyl-3,7-dihydro-purine-2,6-dione), BG 9719 (8-(2S-5,6-exo-epoxy-endo-norborn-2-yl)-1,3-dipropyl-3,7-dihydro-purine-2,6-dione), or BG 9928 (3-[4-(2,6-dioxo-1,3-dipropyl-2,3,6,7-tetrahydro-1H-purin-8-yl)-bicyclo[2.2.2]oct-1-yl]-propionic acid) beginning 10 minutes before the occlusion. All of the antagonists were administered at a dose of 1 mg/kg as an i.v. bolus followed by and infusion of 10 μg/kg/min continued until immediately before reperfusion (70 minutes total).

There were no significant differences between the four groups in systemic hemodynamics (heart rate and blood pressure), maximal left ventricular dP/dt, or regional myocardial blood flow (see Tables 1, 4, and 5), demonstrating that hemodynamic variables were not affected by the antagonists. There were also no differences in the portion of the left ventricle that was subjected to ischemia during coronary occlusion (risk region size; FIG. 1A). However, infarct size expressed as either a percentage of the risk region (FIG. 1B) or as a percentage of the left ventricle (FIG. 1C) was significantly smaller in the two groups of dogs treated with CPX (51% reduction) or BG 9928 (49% reduction)-. Infarct size in the group of dogs treated with BG 9928 was similar to that in the control group. When infarct size expressed as a percentage of the risk region was plotted versus transmural collateral blood flow (FIG. 1D), an inverse relationship was apparent that could be fitted by linear regression analysis. In the CPX-treated and BG 9928-treated groups, this relationship was shifted downward compared to the control group, indicating that infarct size was smaller in these two groups at any given degree of collateral flood flow. The relationship between infarct size and collateral blood flow was similar between the control group and the BG 9719-treated group. Thus, treatment with CPX or BG 9928 (but not treatment with BG 9719) prior to the occlusion resulted in a significant reduction in infarct size that was not related to changes in systemic hemodynamics or regional collateral blood flow.

TABLE 1
Hemodynamic variables from Protocol I (Pretreatment).

| | baseline | occ30′ | occ60′ | rep1 hr | rep2 hr | rep3 hr |
|---|---|---|---|---|---|---|
| Vehicle | | | | | | |
| HR (beats/min) | 155 ± 3 | 153 ± 2 | 154 ± 3 | 154 ± 3 | 152 ± 2 | 152 ± 5 |
| MBP (mmHg) | 107 ± 5 | 105 ± 5 | 102 ± 5 | 104 ± 5 | 110 ± 6 | 109 ± 6 |
| LVdP/dt (mmHg/sec) | 1663 ± 89 | 1650 ± 121 | 1813 ± 119 | 1650 ± 76 | 1538 ± 87 | 1513 ± 75 |
| CPX | | | | | | |
| HR | 150 ± 2 | 153 ± 4 | 152 ± 4 | 150 ± 5 | 153 ± 5 | 151 ± 5 |
| MBP | 90 ± 4 | 94 ± 7 | 98 ± 8 | 97 ± 5 | 102 ± 6 | 105 ± 6 |
| LVdP/dt | 1650 ± 106 | 1481 ± 146 | 1631 ± 92 | 1506 ± 77 | 1538 ± 74 | 1538 ± 135 |
| BG 9719 | | | | | | |
| HR | 155 ± 2 | 161 ± 4 | 159 ± 4 | 157 ± 5 | 160 ± 4 | 161 ± 4 |
| MBP | 104 ± 6 | 109 ± 5 | 103 ± 5 | 106 ± 3 | 114 ± 4 | 112 ± 5 |
| LVdP/dt | 1838 ± 141 | 1931 ± 125 | 1819 ± 205 | 1706 ± 102 | 1781 ± 125 | 1725 ± 113 |
| BG 9928 | | | | | | |
| HR | 152 ± 2 | 150 ± 2 | 151 ± 4 | 153 ± 4 | 153 ± 4 | 154 ± 4 |
| MBP | 87 ± 6 | 92 ± 5 | 95 ± 5 | 87 ± 3 | 97 ± 5 | 99 ± 4 |
| LVdP/dt | 1518 ± 154 | 1631 ± 115 | 1650 ± 136 | 1463 ± 62 | 1463 ± 141 | 1463 ± 84 |

HR, heart rate;
MBP, mean arterial blood pressure;
LVdP/dt, maximal left ventricular dP/dt.

3. Preconditioning Experimental Protocol

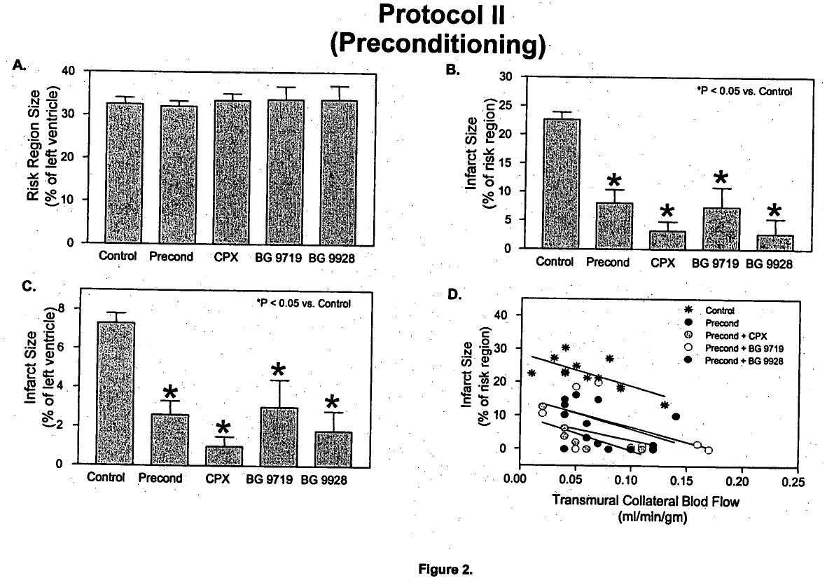

In the preconditioning protocol (see FIG. 2, protocol II) all of the dogs were subjected to 60 minutes of coronary artery occlusion followed by three hours of reperfusion. Preconditioning was elicited by four 5-minute occlusion/5-minute reperfusion cycles produced 10 minutes before the 60-minute occlusion. Four groups of dogs were randomly assigned to receive vehicle, CPX, BG 9719, or BG 9928 beginning 10 minutes before the first preconditioning occlusion. The antagonists were administered at a dose of 1 mg/kg i.v. bolus followed by an infusion of 10 μg/kg/min that was continued until release of the prolonged occlusion (115 minutes total).

Similar to the pretreatment group, there were no significant differences in systemic hemodynamics, regional myocardial blood flow, or risk region sizes between the four groups in the preconditioning protocol (see Tables 2, 4, and 5, FIG. 2A). Preconditioning with four 5 minute-occlusion/5-minute reperfusion cycles before the 60-minute occlusion produced a marked reduction in infarct size (65% reduction) compared to the non-preconditioned control group from Protocol I (FIGS. 2B and 2C). The average infarct sizes (expressed either as a percentage of the risk region or the left ventricle) in the groups of dogs treated with the adenosine receptor antagonists were also significantly smaller compared to the non-preconditioned control group and were similar or slightly smaller than the preconditioned control group (FIGS. 2B and 2C). Preconditioning shifted the relationship between infarct size and collateral blood flow downward compared, to the non-preconditioned control group (FIG. 2D). This relationship was shifted downward further in the groups of dogs treated with CPX or BG9928, but not by BG9719. These results demonstrated that treatment with CPX, BG9719, or BG9928 did not block the protective effects of ischemic preconditioning elicited by multiple occlusion/reperfusion cycles. The results also suggested that treatment with CPX or BG 9928 (but not BG 9719) added to the protective effect of ischemic preconditioning.

TABLE 2
Hemodynamic variables from Protocol II (Preconditioning).

| | Baseline | occ30′ | occ60′ | rep1 hr | rep2 hr | rep3 hr |
|---|---|---|---|---|---|---|
| Vehicle | | | | | | |
| HR (beats/min) | 155 ± 4 | 153 ± 4 | 152 ± 4 | 144 ± 3 | 144 ± 3 | 146 ± 2 |
| MBP (mmHg) | 103 ± 6 | 101 ± 6 | 104 ± 6 | 107 ± 6 | 108 ± 4 | 106 ± 5 |
| LVdP/dt (mmHg/sec) | 1606 ± 196 | 1625 ± 142 | 1550 ± 124 | 1394 ± 94 | 1356 ± 75 | 1281 ± 60 |
| CPX | | | | | | |
| HR | 151 ± 1 | 150 ± 3 | 148 ± 3 | 150 ± 5 | 151 ± 4 | 152 ± 4 |
| MBP | 87 ± 6 | 88 ± 4 | 96 ± 8 | 91 ± 5 | 100 ± 5 | 100 ± 6 |
| LVdP/dt | 1369 ± 140 | 1294 ± 130 | 1388 ± 113 | 1181 ± 82 | 1256 ± 89 | 1313 ± 105 |
| BG 9719 | | | | | | |
| HR | 156 ± 3 | 152 ± 4 | 152 ± 5 | 155 ± 7 | 156 ± 6 | 156 ± 6 |
| MBP | 105 ± 7 | 103 ± 5 | 103 ± 5 | 97 ± 6 | 99 ± 6 | 101 ± 5 |
| LVdP/dt | 1693 ± 121 | 1671 ± 111 | 1736 ± 130 | 1500 ± 164 | 1457 ± 153 | 1479 ± 155 |
| BG 9928 | | | | | | |
| HR | 149 ± 1 | 149 ± 2 | 150 ± 1 | 149 ± 1 | 148 ± 1 | 148 ± 1 |
| MBP | 86 ± 2 | 84 ± 3 | 84 ± 3 | 80 ± 5 | 87 ± 5 | 86 ± 3 |
| LVdP/dt | 1300 ± 50 | 1400 ± 74 | 1375 ± 72 | 1100 ± 50 | 1125 ± 64 | 1175 ± 72 |

HR, heart rate;
MBP, mean arterial blood pressure;
maximal LVdP/dt, left ventricular dP/dt.

4. Reperfusion Experimental Protocol

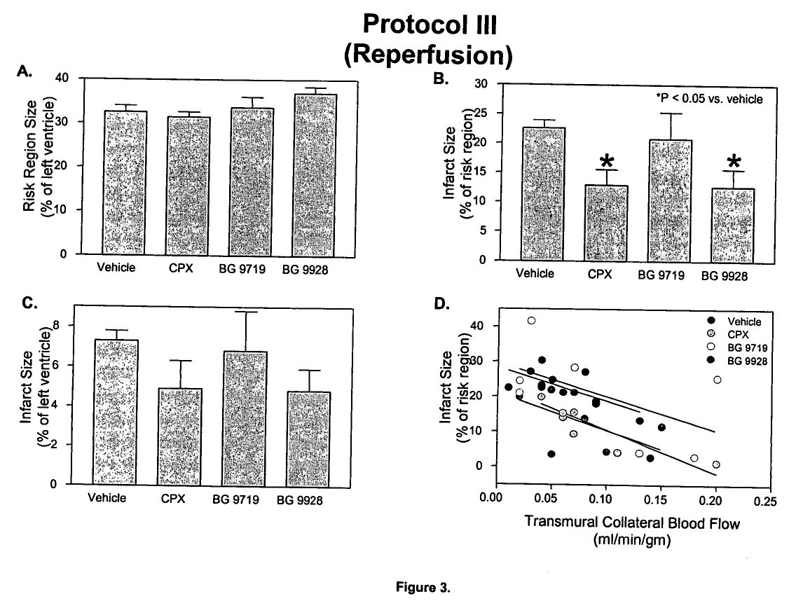

In the reperfusion protocol (see FIG. 3, protocol III), the dogs were subjected to 60 minutes of coronary artery occlusion followed by three hours of reperfusion. Four groups of dogs were randomly assigned to receive vehicle, CPX, BG 9719, or BG 9928 beginning 10 minutes before the release of the occlusion. The antagonists were administered at a dose of 1 mg/kg i.v. bolus followed by an infusion of 10 μg/kg/min for one hour.

There were no significant differences in hemodynamic variables, regional myocardial blood flow, or risk region sizes between the four groups of dogs in this experimental protocol (see Tables 3-5 and FIG. 3A).

Infarct size expressed as a percentage of the risk region was reduced significantly by administration of CPX or BG 9928 during the early phase of reperfusion (FIG. 3B). However, administration of BG 9719 had no protective effect. The relationship between infarct size and collateral blood flow was shifted downward in the two groups of dogs treated with CPX or BG 9928 compared to the control group (FIG. 3C). The reduction in infarct size produced by CPX and BG 9928 in this protocol was smaller in magnitude (42% and 44%, respectively) compared to Protocol I when they were administered prior to ischemia and a significant reduction in infarct size was not observed when the data was expressed as a percentage of the entire left ventricle (FIG. 3D), perhaps due to the small number of animals studied. These data demonstrated that CPX and BG 9928 (but not BG 9719) reduced infarct size when administered at the time of reperfusion.

TABLE 3
Hemodynamic variables from Protocol III (Reperfusion).

| | baseline | occ30′ | occ60′ | rep1 hr | rep2 hr | rep3 hr |
|---|---|---|---|---|---|---|
| Vehicle | | | | | | |
| HR (beats/min) | 155 ± 3 | 153 ± 2 | 154 ± 3 | 154 ± 3 | 152 ± 2 | 152 ± 5 |
| MBP (mmHg) | 107 ± 5 | 105 ± 5 | 102 ± 5 | 104 ± 5 | 110 ± 6 | 109 ± 6 |
| LVdP/dt (mmHg/sec) | 1663 ± 89 | 1650 ± 121 | 1813 ± 119 | 1650 ± 76 | 1538 ± 87 | 1513 ± 75 |
| CPX | | | | | | |
| HR | 150 ± 2 | 149 ± 1 | 151 ± 1 | 152 ± 3 | 151 ± 4 | 156 ± 4 |
| MBP | 102 ± 4 | 99 ± 7 | 105 ± 6 | 108 ± 5 | 112 ± 4 | 114 ± 4 |
| LVdP/dt | 1556 ± 85 | 1531 ± 159 | 1688 ± 105 | 1688 ± 97 | 1650 ± 57 | 1631 ± 72 |
| BC 9719 | | | | | | |
| HR | 150 ± 3 | 154 ± 3 | 153 ± 4 | 154 ± 5 | 155 ± 6 | 151 ± 4 |
| MBP | 102 ± 5 | 95 ± 7 | 101 ± 5 | 101 ± 3 | 103 ± 3 | 97 ± 5 |
| LVdP/dt | 1519 ± 125 | 1400 ± 149 | 1569 ± 165 | 1500 ± 102 | 1425 ± 85 | 1350 ± 90 |
| BG 9928 | | | | | | |
| HR | 151 ± 1 | 151 ± 3 | 150 ± 2 | 147 ± 2 | 148 ± 2 | 150 ± 3 |
| MBP | 90 ± 6 | 90 ± 5 | 96 ± 4 | 88 ± 5 | 92 ± 5 | 95 ± 4 |
| LVdP/dt | 1594 ± 106 | 1638 ± 132 | 1744 ± 69 | 1406 ± 49 | 1463 ± 74 | 1463 ± 79 |

HR, heart rate;
MBP, mean arterial blood pressure;
maximal LVdP/dt, left ventricular dP/dt.

TABLE 4
Regional myocardial blood flow data (ml/min/gm) from Protocols I, II, and III in the non-
ischemic region (region perfused by the left circumflex coronary artery).

| | Protocol I | | Protocol II | | Protocol III | |
|---|---|---|---|---|---|---|
| | occ30 | rep3 hr | occ30 | rep3 hr | occ30 | rep3 hr |
| Vehicle | | | | | | |
| epi | 0.65 ± 0.06 | 0.53 ± 0.05 | 0.66 ± 0.06 | 0.69 ± 0.10 | 0.65 ± 0.06 | 0.53 ± 0.05 |
| mid | 0.75 ± 0.09 | 0.60 ± 0.05 | 0.62 ± 0.07 | 0.57 ± 0.09 | 0.75 ± 0.09 | 0.60 ± 0.05 |
| endo | 0.76 ± 0.09 | 0.69 ± 0.09 | 0.61 ± 0.10 | 0.59 ± 0.11 | 0.76 ± 0.09 | 0.69 ± 0.09 |
| trans | 0.72 ± 0.07 | 0.61 ± 0.05 | 0.63 ± 0.07 | 0.62 ± 0.05 | 0.72 ± 0.07 | 0.61 ± 0.05 |
| CPX | | | | | | |
| epi | 0.60 ± 0.08 | 0.66 ± 0.07 | 0.97 ± 0.20 | 0.85 ± 0.12 | 0.69 ± 0.05 | 0.96 ± 0.12 |
| mid | 0.66 ± 0.08 | 0.64 ± 0.07 | 0.78 ± 0.12 | 0.76 ± 0.12 | 0.67 ± 0.07 | 0.94 ± 0.12 |
| endo | 0.54 ± 0.04 | 0.61 ± 0.06 | 0.73 ± 0.22 | 0.81 ± 0.15 | 0.71 ± 0.07 | 1.02 ± 0.12 |
| transmural | 0.60 ± 0.06 | 0.64 ± 0.06 | 0.83 ± 0.20 | 0.81 ± 0.13 | 0.69 ± 0.06 | 0.97 ± 0.11 |
| B 9719 | | | | | | |
| epi | 0.70 ± 0.08 | 0.64 ± 0.09 | 0.91 ± 0.22 | 0.83 ± 0.13 | 0.60 ± 0.08 | 0.46 ± 0.03 |
| mid | 0.77 ± 0.06 | 0.64 ± 0.07 | 0.92 ± 0.14 | 0.87 ± 0.11 | 0.66 ± 0.06 | 0.50 ± 0.02 |
| endo | 0.77 ± 0.08 | 0.67 ± 0.08 | 0.86 ± 0.16 | 0.88 ± 0.20 | 0.63 ± 0.06 | 0.59 ± 0.06 |
| transmural | 0.75 ± 0.07 | 0.65 ± 0.08 | 0.90 ± 0.13 | 0.86 ± 0.12 | 0.63 ± 0.05 | 0.52 ± 0.03 |
| BG 9928 | | | | | | |
| epi | 0.87 ± 0.08 | 0.73 ± 0.07 | 0.48 ± 0.14 | 0.45 ± 0.06 | 0.83 ± 0.07 | 0.84 ± 0.10 |
| mid | 0.80 ± 0.07 | 0.71 ± 0.07 | 0.49 ± 0.14 | 0.47 ± 0.12 | 0.87 ± 0.06 | 0.89 ± 0.08 |
| endo | 0.80 ± 0.11 | 0.79 ± 0.06 | 0.51 ± 0.12 | 0.56 ± 0.14 | 0.85 ± 0.06 | 0.88 ± 0.08 |
| transmural | 0.82 ± 0.06 | 0.74 ± 0.06 | 0.49 ± 0.13 | 0.50 ± 0.13 | 0.85 ± 0.05 | 0.87 ± 0.08 |

epi, epicardium;
mid, midmyocardium;
endo, endocardium;
trans, transmural

TABLE 5
Regional myocardial blood flow data (ml/min/gm) from Protocols I, II, and III in the
ischemic-reperfused region (region perfused by the left anterior descending coronary
artery).

| | Protocol I | | Protocol II | | Protocol III | |
|---|---|---|---|---|---|---|
| | occ30 | rep3 hr | occ30 | rep3 hr | occ30 | rep3 hr |
| Vehicle | | | | | | |
| epi | 0.08 ± 0.01 | 0.47 ± 0.10 | 0.10 ± 0.04 | 0.48 ± 0.12 | 0.08 ± 0.01 | 0.47 ± 0.10 |
| mid | 0.06 ± 0.01 | 0.50 ± 0.08 | 0.06 ± 0.02 | 0.35 ± 0.04 | 0.06 ± 0.01 | 0.50 ± 0.08 |
| endo | 0.05 ± 0.01 | 1.01 ± 0.16 | 0.07 ± 0.02 | 1.06 ± 0.13 | 0.05 ± 0.01 | 1.01 ± 0.16 |
| trans | 0.06 ± 0.01 | 0.66 ± 0.10 | 0.08 ± 0.02 | 0.63 ± 0..04 | 0.06 ± 0.01 | 0.66 ± 0.10 |
| CPX | | | | | | |
| epi | 0.15 ± 0.04 | 0.48 ± 0.06 | 0.07 ± 0.03 | 0.62 ± 0.12 | 0.10 ± 0.01 | 0.50 ± 0.04 |
| mid | 0.08 ± 0.02 | 0.49 ± 0.04 | 0.05 ± 0.01 | 0.54 ± 0.11 | 0.07 ± 0.01 | 0.40 ± 0.04 |
| endo | 0.05 ± 0.01 | 0.90 ± 0.16 | 0.04 ± 0.01 | 0.68 ± 0.12 | 0.04 ± 0.01 | 0.93 ± 0.15 |
| transmural | 0.09 ± 0.02 | 0.62 ± 0.06 | 0.06 ± 0.02 | 0.61 ± 0.10 | 0.07 ± 0.01 | 0.61 ± 0.05 |
| B 9719 | | | | | | |
| epi | 0.11 ± 0.03 | 0.44 ± 0.10 | 0.14 ± 0.04 | 0.63 ± 0.12 | 0.10 ± 0.03 | 0.31 ± 0.04 |
| mid | 0.06 ± 0.02 | 0.31 ± 0.04 | 0.08 ± 0.02 | 0.43 ± 0.04 | 0.07 ± 0.03 | 0.33 ± 0.05 |
| endo | 0.05 ± 0.01 | 0.77 ± 0.19 | 0.06 ± 0.01 | 0.64 ± 0.10 | 0.04 ± 0.01 | 0.72 ± 0.13 |
| transmural | 0.09 ± 0.02 | 0.51 ± 0.10 | 0.09 ± 0.02 | 0.56 ± 0.10 | 0.09 ± 0.03 | 0.45 ± 0.06 |
| BG 9928 | | | | | | |
| epi | 0.14 ± 0.05 | 0.48 ± 0.11 | 0.12 ± 0.04 | 0.45 ± 0.13 | 0.10 ± 0.02 | 0.66 ± 0.12 |
| mid | 0.09 ± 0.03 | 0.39 ± 0.05 | 0.06 ± 0.01 | 0.31 ± 0.10 | 0.08 ± 0.02 | 0.67 ± 0.15 |
| endo | 0.05 ± 0.01 | 0.73 ± 0.12 | 0.03 ± 0.01 | 0.72 ± 0.30 | 0.05 ± 0.01 | 1.20 ± 0.15 |
| transmural | 0.09 ± 0.03 | 0.54 ± 0.06 | 0.07 ± 0.01 | 0.49 ± 0.14 | 0.08 ± 0.02 | 0.84 ± 0.12 |

epi, epicardium;
mid, midmyocardium;
endo, endocardium;
trans, transmural

TABLE 6
Dissociation constants of antagonist for recombinant canine A₁, A₂ₐ, and
A₃ adenosine receptors determined by radioligand binding analysis.

| Compound | A₁ | A₂ₐ | A₃ |
|---|---|---|---|
| CPX | 18.1 ± 4.4 | 162 ± 22 | 1,960 ± 420 |
| BG 9719 | 35.8 ± 4.0 | 2,820 ± 268 | 19,070 ± 540 |
| BG 9928 | 28.9 ± 4.1 | 4,307 ± 1,230 | 37,670 ± 9,030 |

Kᵢ values (nM ± SEM; n = 3) obtained from competition binding experiments with membranes from transfected HEK 293 cells using ³H-CPX, ³H-ZM 241385, and ³R-PIA as the radioligand for A₁, A₂ₐ, and A₃ receptors, respectively.

5. Membrane Preparation

HEK 293 (Human Embroynic Kidney) membranes expressing human A_{2b} adenosine receptors were purchased from Receptor Biology; HEK 293 cell membranes expressing human A₂ₐ receptors were purchased from PerkinElmer (Boston, Mass.); CHO-K1 cell membranes expressing human A₁ receptors and HEK 293 cell membranes expressing human A₃ receptors were made from the corresponding stably transfected cells established in house.

6. Radioligand Binding Assays

Membranes (40-70 μg membrane protein), radioligands, and varying concentrations of competing ligands were incubated in triplicate in 0.1 ml buffer HE plus 2 units/mL adenosine deaminase for 2.5 hours at 21° C. The radioligands used for competitive binding assays were: [³H]-8-cyclopentyl-1,3-dipropyxanthine ([³H]-DPCPX) (NEN, Boston, Mass.) for A₁ and A_{2b} adenosine receptors, [³H]-4-(2-[7-amino-2-(furyl)(1,2,4)triazole(2,3-a)(1,3,5)triazin-5-ylaminoethyly)phenol ([³H] ZM241385) for A₂ₐ adenosine receptors (Tocris, Bristol, UK), and [¹²⁵Iodine]-labeled N6-(4-aminobenzyl)-9-(5-(methylcarbonyl)-β-D-ribofuranosyl) adenine ([¹²⁵I]-AB-MECA) or [³H]-R-N⁶-phenylisopropyladenosine (H³R]-R-PIA) for A₃ adenosine receptors (both from NEN, Boston, Mass.). Nonspecific binding was measured in the presence of 10 μM 5′N ethylcarboxamidoadenosine (NECA, from RBI-Sigma, Natick, Mass.) for A₁ and A_{2b} receptors, or 10 μM xanthine amino congener (XAC, from RBI-Sigma, Natick, Mass.) for A₂ₐ receptors. Binding assays were terminated by filtration over Whatman GF/C glass fiber filters using a BRANDEL cell harvester (Gaithersburg, Md.). The-filters were rinsed three times with 3-4 mL ice-cold 10 mM Tris-HCl, pH 7.4 and 5 mM magnesium chloride (MgCl₂) at 4° C., and were counted in a Wallac β-counter (Perkin Elmer, Boston, Mass.).

TABLE 7
K_{I} Values (nM) or Percent (%) Inhibition at 10 μM
Antagonist in Radioligand Competitive
Binding Assays

K_{I} (nM) or Percent (%) Inhibition at 10 μM
Antagonist in Radioligand Competitive
Binding Assays
Adenosine Receptor

| Species | A₁ | A₂ₐ | A_{2b} | A₃ |
|---|---|---|---|---|
| BG9928 | 12.2 | 4059 | 88.53 ± 21.03^{a} | 30%^{b} |
| DPCPX | 5.3 | 156^{c} | 56 | 262 |
| BG9719 | 10.3 | 9152 | 853 ± 270^{a} | 40.6% |

ND: Not done
^{a}N = 3
^{b}Percent inhibition at 10 μM BG9928.
^{c}See J. Linden, Annu. Rev. Pharmacol. Toxicol., 41, pp. 775-787 (2001).

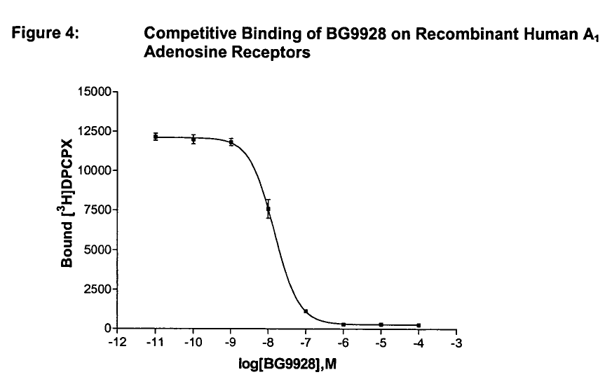

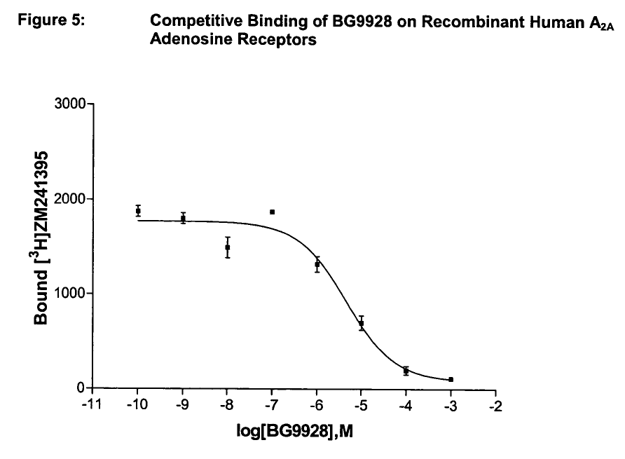

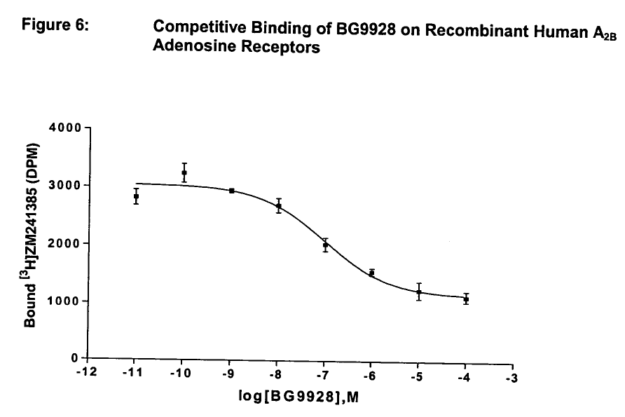

The K_{I} values for BG9928, DPCPX and BG9717 were 12.2 nM, 5.3 nM and 10.3 nM, respectively, in competitive binding assays with recombinant human A₁ adenosine receptors and [³H]-DPCPX as the radioligand (see Table 7, FIG. 4). The K, values for BG9928, DPCPX and BG9717 were 4059 nM, 156 nM and 9152 nM, respectively, in competitive binding assays with recombinant human A₂ₐ adenosine receptors and [³H]-ZM241385 as the radioligand (see Table 7, FIG. 5). The K_{I} value for BG9928, DPCPX and BG9717 was 88.53±21.03 nM (N=3), 56 nM and 853±270 nM (N=3), respectively, in competitive binding with recombinant human A_{2b} adenosine receptors and [³H]-ZM241385 as the radioligand (see Table 7, FIG. 6).

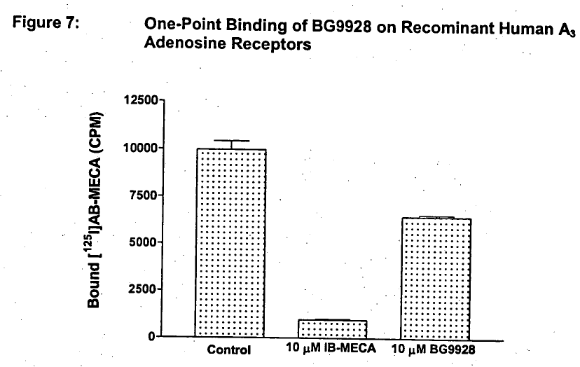

One-point binding assays were performed to determine the effect of 10 μM BG9928 on the binding of [¹²⁵I]-AB-MECA to recombinant human A₃ adenosine receptor membranes. In a one-point binding assay with recombinant human A₃ adenosine receptors, 10 μM BG9928 resulted in 30% inhibition of [³H]-ZM241385 binding (FIG. 7).

7. Radioligand Binding Assay

Membranes (50 μg membrane protein), radioligands, and varying concentrations of competing ligands were incubated in triplicate in 0.1 mL buffer HE plus 2 units/mL adehosine, deaminase for 2 hours at 21° C. The radioligand used for competitive binding assays for human A_{2B} adenosine receptors was [³H]-8-cyclopentyl-1,3-dipropyxanthine ([³H]-DPCPX, 30-40 nM) (NEN, Boston, Mass.). Nonspecific binding was measured in the presence of 10 μM 5′N-ethylcarboxamidoadenosine (NECA; from RBI-Sigma, Natick, Mass.). Binding assays were terminated by filtration over Whatman GF/C glass fiber filters using a BRANDEL cell harvester (Gaithersburg, Md.). The filters were rinsed three times with 3 to 4 mL ice-cold 10 mM Tris-HCl, pH 7.4 and 5 mM magnesium chloride (MgCl₂) at 4° C. and were counted in a Wallac β-counter (Perkin Elmer, Boston, Mass.).

Competitive binding data were fit to a single site binding model and plotted using Prizm GraphPad. The Cheng-Prusoff equation K_{I}=IC₅₀/(1+[I]/K_{D}) was used to calculate K_{I} values from IC₅₀ values, where K_{I} is the affinity constant for the competing ligand, [I] is the concentration of the free radioligand, and K_{D} is the affinity constant for the radioligand (Cheng and Prusoff 1973). The K_{I} values of several compounds of this invention-are provided in Table 8.

TABLE 8
K_{I} (nM) in Radioligand Competitive Binding Assays

| Compound | | | |
|---|---|---|---|
| No. | Structure | Assay | Ki (nM) |
| 1 | 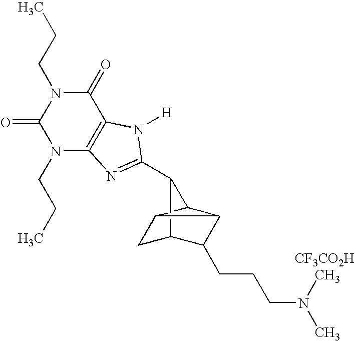 | ADENOSINE A2B (HEK293) | 14.6 |
| 2 | 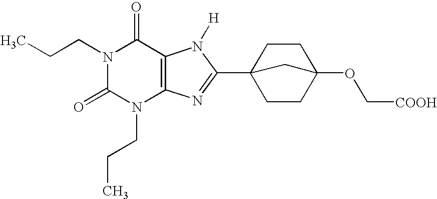 | ADENOSINE A2B (HEK293) | 15.2 |
| 3 | 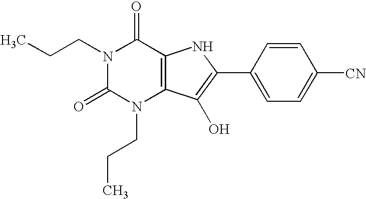 | ADENOSINE A2B (HEK293) | 27 |
| 4 | 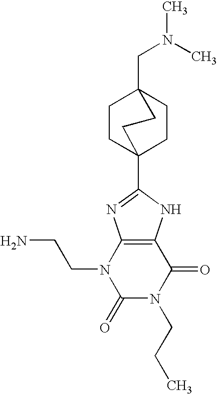 | ADENOSINE A2B (HEK293) | 39 |
| 5 | 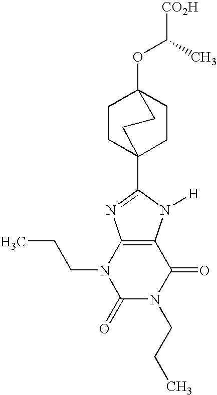 | ADENOSINE A2B (HEK293) | 45.1 |
| 6 | 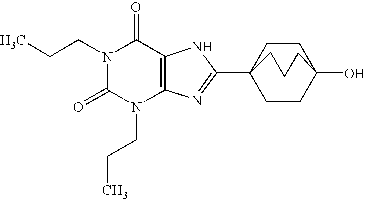 | ADENOSINE A2B (HEK293) | 47 |
| 7 | 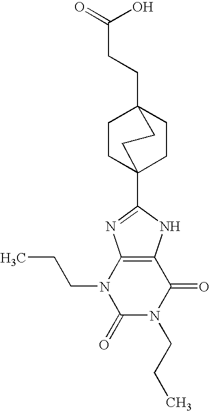 | ADENOSINE A2B (HEK293) | 51.4 |
| 8 | 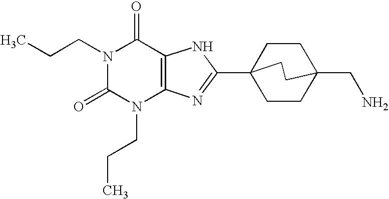 | ADENOSINE A2B (HEK293) | 55.2 |
| 9 | 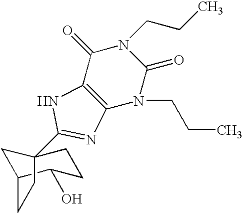 | ADENOSINE A2B (HEK293) | 76.9 |
| 10 | 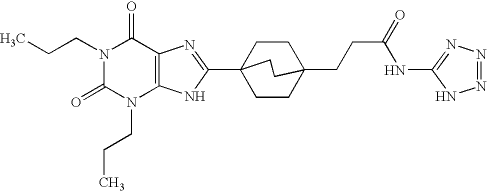 | ADENOSINE A2B (HEK293) | 85.6 |
| 11 | 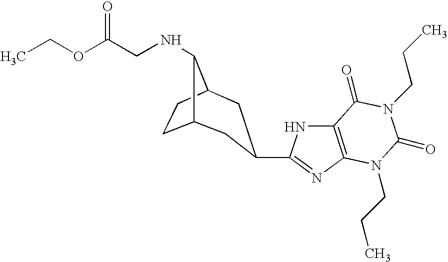 | ADENOSINE A2B (HEK293) | 93 |
| 12 | 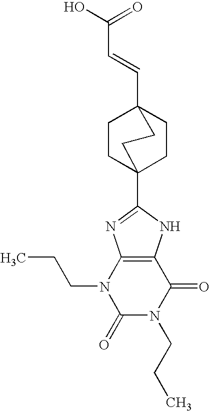 | ADENOSINE A2B (HEK293) | 100 |
| 13 | 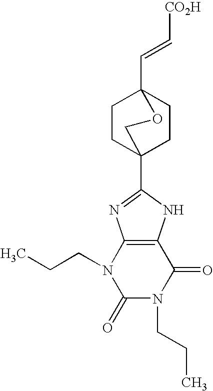 | ADENOSINE A2B (HEK293) | 117 |
| 14 | 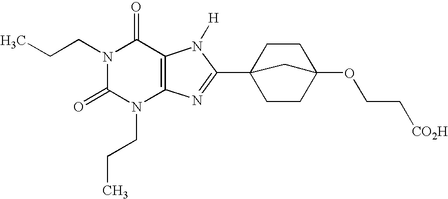 | ADENOSINE A2B (HK293) | 125 |
| 15 | 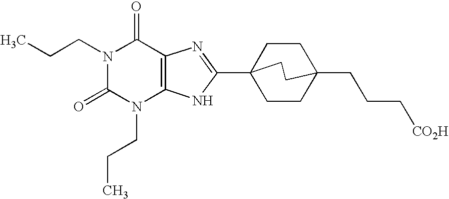 | ADENOSINE A2B (HEK293) | 127 |
| 16 | 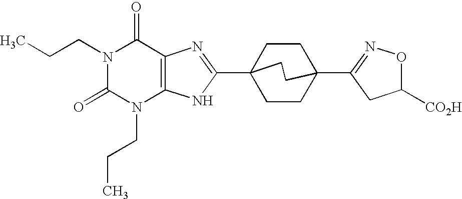 | ADENOSINE A2B (HEK293) | 131 |
| 17 | 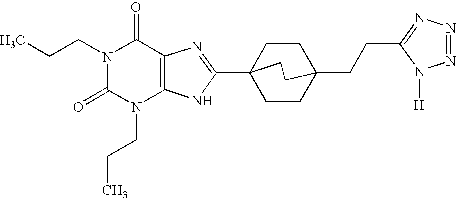 | ADENOSINE A2B (HEK293) | 159.62 |
| 18 | 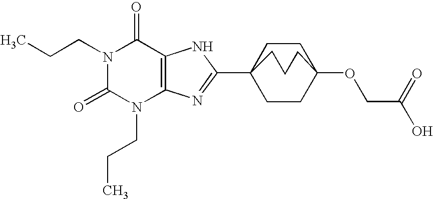 | ADENOSINE A2B (HEK293) | 168 |

8. Flourescence Imaging Plate Reader (FLIPR) Functional Assays

Flourescence imaging plate reader (FLIPR) assays for the determination of calcium were performed with HEK 293 cells which exhibit stable expression of human and rat A_{2b} adenosine receptors and CHO-K1 cells that exhibit stable expression of recombinant human A₁ adenosine receptors. Cells were seeded into 96-well tissue culture plates with black walls and clear bottoms, and cultured to an 80-0.90% confluent monolayer. Without removing the media, an equal volume of dye (from calcium assay kit purchased from Molecular Devices) was added. Cell plates were incubated for 1 hour at 37° C. and were then transferred to the-FLIPR unit (Molecular Devices).

For assay of recombinant human A₁ adenosine receptors, CHO-K1 cells were incubated with increasing doses of agonist (N-6-cyclopentyladenosine, CPA) to determine the concentration of agonist that produced 50% of a maximum response. This concentration of agonist (200 nM CPA) was then incubated with increasing concentrations (10⁻¹² M to 10⁻⁵ M) of antagonist, BG9928. For assay of recombinant human and rat A_{2b} adenosine receptors, HEK-293 cells were incubated with increasing doses of agonist (5′N-ethylcarboxamidoadenosine, NECA) to determine the concentration of agonst that produced 50% of a maximum response. This concentration of agonist (5 μM NECA for human A_{2b} receptors) or varying concentrations (for rat A_{2b} receptors) were then incubated with increasing concentrations of antagonist, BG9928 (10⁻¹² M to 5×10⁻⁶ M for human A_{2b} recptors and 10, 1100, or 300 nM for rat A_{2b} receptors).

The FLIPR integrates an argon laser excitation source, a 96-well pipettor, and a detection system utilizing a CCD (Charged Coupled Device) imaging camera. Fluorescence emissions from the 96 wells were monitored simultaneously at excitation and emission wavelength of 488 and 520 nm, respectively. Fluorescence data were collected at 0.1-sec intervals before and after simultaneous rapid addition of compounds to the 96-well plate. Results were read as relative fluorescence units (RFU).

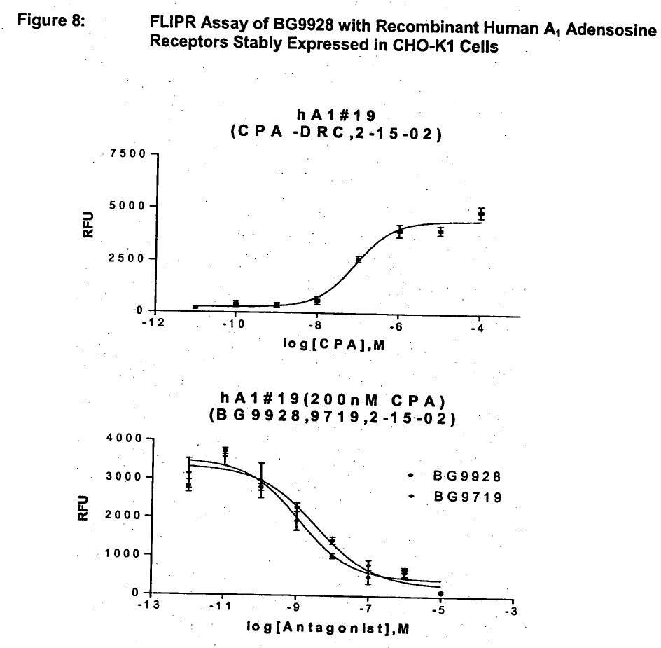

FLIPR functional assays were performed with BG9928 using recombinant human A₁ adenosine receptors, which were stably expressed in CHO-K1 cells. The antagonist dissociation constant (KB) for BG9928 and BG9719 was 0.60 nM and 0.46 nM, respectively on recombinant human A₁ adenosine receptor using null methodology (see Table 9 and FIG. 8).

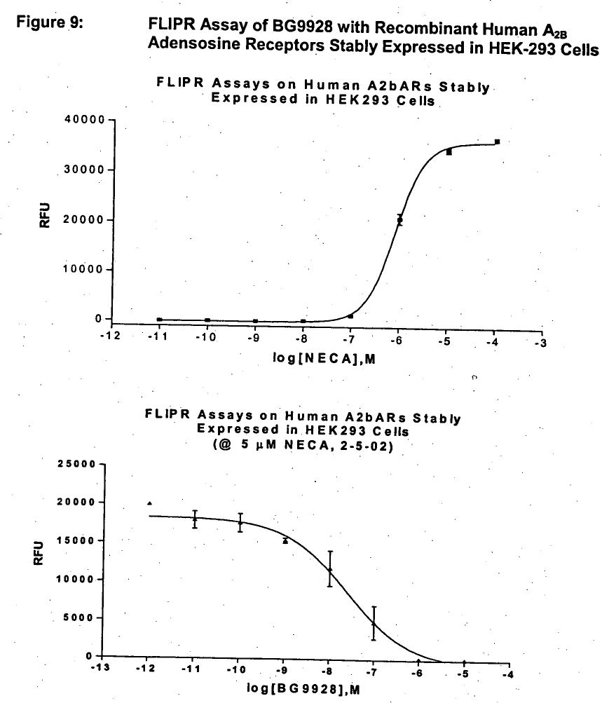

FLIPR functional assays were performed with BG9928 using recombinant human A_{2b} adenosine receptors, which were stably expressed in HEK293 cells. The antagonist K_{B} for BG9928, BG9719 and DPCPX was 3.36 nM, 182 nM and 23.6 nM, respectively, on recombinant human A_{2b} adenosine receptors using null methodology (see Table 9 and FIG. 9).

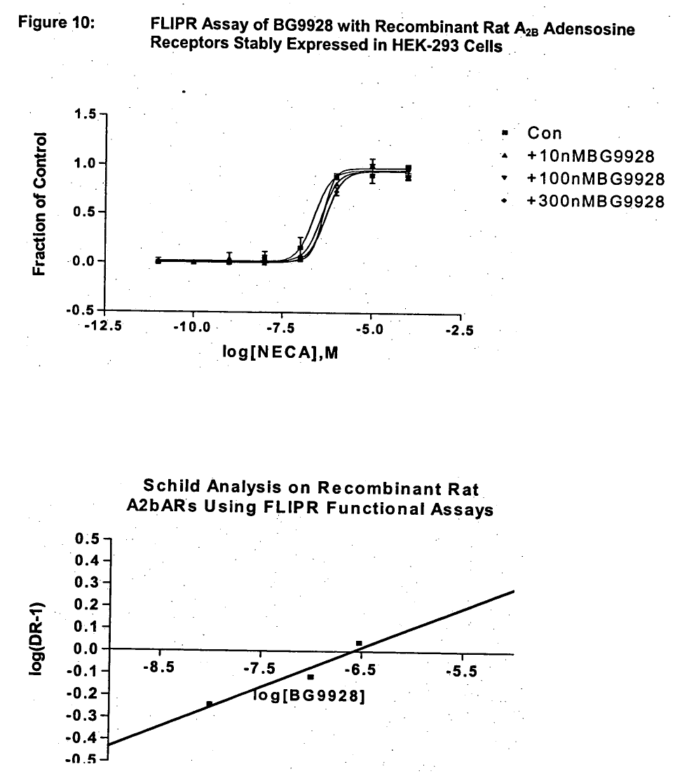

FLIPR functional assays were performed with BG9928; using recombinant rat. A_{2b} adenosine receptors, which were stably expressed in HEK293 cells. The antagonist KB for BG9928 was 257 nM using null methodology and the pA₂ was 6.59 using Schild analysis (see Table 9 and FIG. 10).

TABLE 9
Summary of K_{B} (nM) Values for Antagonists in FLIPR
Functional Assays (Human Receptor Subtypes)

K_{B} (nM) for Antagonists in FLIPR
Functional Assays
Adenosine Receptor

| Species | A₁ | A₂ₐ | A_{2b} | A₃ |
|---|---|---|---|---|
| BG9928 | 0.60 | ND | 3.36 | ND |
| BG9719 | 0.46 | ND | 182 | ND |
| DPCPX | ND | ND | 23.6 | ND |

ND: Not done

9. Data Analysis

Data are presented as mean standard error of the mean (SEM) or standard deviation (SD). Saturation data were analyzed using Marquardt's5 non-linear least squares methods and plotted using Prizm GraphPad. Competitive binding data were fit to a single site binding model and plotted using Prizm GraphPad. The Cheng-Prusoff equation K_{I}=IC50/(1+[I]/K_{D}) was used to calculate K_{I} values from IC₅₀ values, where K_{I} is the affinity constant for the competing ligand, [I] is the concentration of the free radioligand, and K_{D} is the affinity constant for the radioligand (Cheng and Prusoff 1973).

In FLIPR functional assays, agonist concentration-response curves were fitted to a logistic equation by use of the nonlinear regression program in Prizm GraphPad. Antagonist dissociation constants (K_{B}) was estimated Using the null method developed by Lazareno and Roberts (1987). A Schild analysis was performed to estimate the potency of the compounds as antagonists (pA₂). PA₂ is the negative log of the concentration of antagonist that could produce a 2-fold shift in the concentration-response curve, where response was defined as 50% of the maximum response.

1. A method of preventing, limiting, or treating, ischemia reperfusion injury in a mammal, comprising:
identifying a mammal that has undergone an ischemic event, or in which an ischemic event is imminent; and
administering a therapeutically effective or prophylactically effective amount of an A_{2b} adenosine receptor antagonist to the mammal within ten days before or after the ischemic event;
wherein the A_{2b} adenosine receptor antagonist is a compound of formula (I), 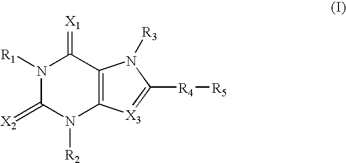
or a pharmaceutically acceptable salt or N-oxide thereof, wherein:
each of R₁, R₂, and R₃, independently, is:
a) hydrogen;
b) C₁₋₆ alkyl, C₂₋₆ alkenyl, or C₂₋₆ alkynyl; wherein said alkyl, alkenyl, or alkynyl is either unsubstituted or substituted with one or more substituents selected from the group consisting of hydroxy, alkoxy, amino, monoalkylamino, dialkylamino, cycloalkyl, aryl, heterocyclyl, aralkyl, heterocyclylalkyl, acylamino, alkylaminocarbonyl, alkylsulfonylamino, and alkylaminosulfonyl;
c) substituted or unsubstituted aryl; or
d) substituted or unsubstituted heterocyclyl;
R₄ is a single bond, —O—, —(CH₂)₁₃—, —O(CH₂)₁₋₂—, —CH₂OCH₂—, —(CH₂)₁₋₂O—, —CH═CHCH₂—, —CH═CH—, or —CH₂CR═CH—;
R₅ is:
(a) phenyl, or
(b) a bicyclic or tricyclic group selected from the group consisting of: 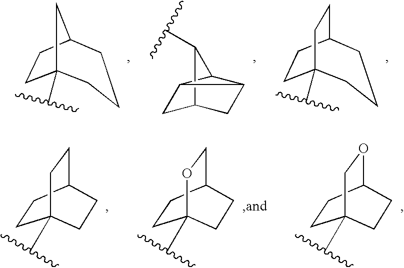
wherein the phenyl, bicyclic, or tricyclic group is either unsubstituted or substituted with one or more Rₐ groups, which is selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino)(R_{b})acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phosphono, substituted or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl(alkyl)amino-, R_{b}-alkyl (alkyl)carbamoyl-, R_{b}-, alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl;
R_{b} is selected from the group consisting of —COOH, —C(CF₃)₂OH, —CONHNHSO₂CF₃, —CONHOR_{c}, —CONHSO₂R_{c}, —CONHSO₂NHR_{c}, —C(OH)R_{c}PO₃H₂, —NHCOCF₃, —NHCONHSO₂R_{c}, —NHPO₃H₂, —NHSO₂R_{c}, —NHSO₂NHCOR_{c}, —OPO₃H₂, —OSO₃H, —PO(OH)R_{c}, —PO₃H₂, —SO₃H, —SO₂NHR_{c}, —SO₃NHCOR_{c}, —SO₃NHCONHCO₂R_{c}, and the following: 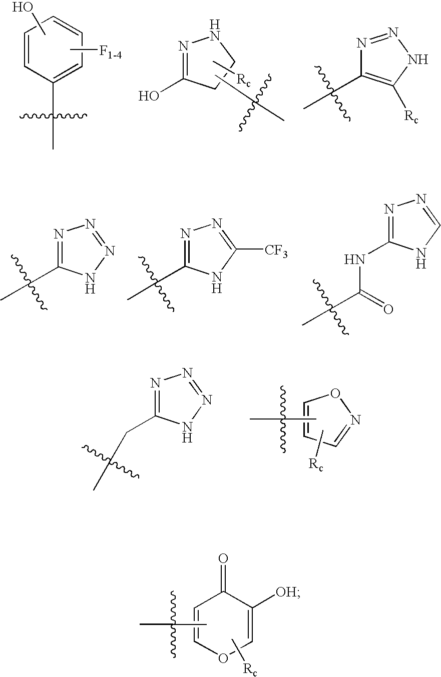
R_{c} is selected from the group consisting of; hydrogen, —C₁₋₄ alkyl, —C₁₋₄ alkyl-CO₂H, and phenyl, wherein the —C₁₋₄ alkyl, —C₁₋₄ alkyl-CO₂H, and phenyl groups are either unsubstituted or substituted with one to three substituents selected from the group consisting of halogen, —OH, —OMe, —NH₂, —NO₂, unsubstituted benzyl, and benzyl substituted with one to three substituents selected from the group consisting of halogen, —OH, —OMe, —NH₂, and —NO₂;
X₁ and X₂ are independently selected from the group, consisting of O and S; and
X₃ is N or CR_{d} wherein R_{d} is selected from the group consisting of:
a) hydrogen;
b) C₁₋₆ alkyl, C₂₋₆ alkenyl, or C₂₋₆ alkynyl; wherein said alkyl, alkenyl, or alkynyl is either unsubstituted or substituted with one or more substituents selected from the group consisting of hydroxy, alkoxy, amino, monoalkylamino, dialkylamino, cycloalkyl, aryl; heterocyclyl, aralkyl, heterocyclylalkyl, acylamino, alkylaminocarbonyl, alkylsulfonylamino, and alkylaminosulfonyl;
c) substituted or unsubstituted aryl; and d) substituted or unsubstituted heterocyclyl.

2. The method of claim 1, wherein R, is C₁₋₆ alkyl.

3. The method of claim 1, wherein R₂ is C₁₋₆ alkyl.

4. The method of claim 1, wherein R₃ is hydrogen.

5. The method of claim 1, wherein R₄ is a single bond.

6. The method of claim 1, wherein R₅ is phenyl substituted with Rₐ.

7. The method of claim 1, wherein R₅ is a substituted bicyclic or tricyclic group selected from the group consisting of:

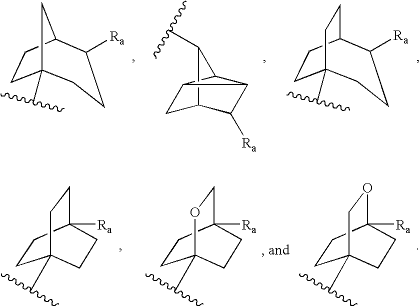

8. The method of claim 1, wherein R₅ is 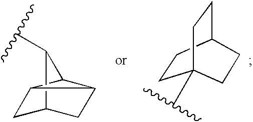
wherein said R₅ is either unsubstituted or substituted with one or more Rₐ groups selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino) (R_{b})acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phospheno, substituted or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl(alkyl)amino-, R_{b}-alkyl(alkyl)carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, -aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl.

9. The method of claim 1, wherein Rₐ is selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino) (R_{b})acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phosphono, substituted aralkylamino, substituted arylcarboxyalkoxycarbonyl, substituted heteroarylsulfonylamino, substituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonyl amino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkyl(alkyl)amino-, R_{b}-alkyl(alkyl)carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, cyano, cycloalkylamino, dialkylaminoalkylcarbamoyl, halogen, heterocyclylalkylamino, hydroxy, oximino, phosphate, substituted aralkylamino, substituted heterocyclyl, substituted heterocyclylsulfonylamino, sulfoxyacylamino, and thiocarbamoyl.

10. The method of claim 1, wherein Rₐ is selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, R_{b}-, R_{b}-alkoxy-, and substituted or unsubstituted heterocyclyl; and
(b) alkoxycarbonylalkylamino, cyano, and hydroxy.

11. The method of claim 1, wherein X₁ is O.

12. The method of claim 1, wherein X₂ is O.

13. The method of claim 1, wherein X₃ is N.

14. The method of claim 1, wherein each of R₁ and R₂ is C₂₋₄ alkyl; R₃ is hydrogen; R₄ is a single bond;
each of X₁ and X₂ is O; and X₃ is N.

15. The method of claim 14, wherein. R₅ is phenyl substituted with Rₐ.

16. The method of claim 15, wherein Rₐ is selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, R_{b}-, and R_{b}-alkoxy-; and
(b) alkoxycarbonylalkylamino, R_{b}-alkoxy-, cyano, substituted or unsubstituted heterocyclyl, and hydroxy.

17. The method of claim 16, wherein Rₐ is cyano.

18. The method of claim 14, wherein R₅ is 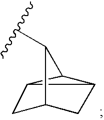
wherein said R₅ is either unsubstituted or substituted with one or more Rₐ groups selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino)(R_{b})acylhydrazinylcarbonyl-, (amino)(R_{b})acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphono, haloalkylsulfonylamino, heterocyclylalkylamino, heterocyclylcarbamoyl, hydroxy, hydroxyalkylsulfonylamino, oximino, phosphono, substituted or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl, aminocycloalkylcarbamoyl, aralkoxycarbonylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl-(alkyl)amino-, R_{b}-alkyl(alkyl)carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, aminoalkylaminocarbonyl, dialkylaminoalkylamino, alkylaminoalkylamino, cyano, cycloalkylamino, dialkylaminoalkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl.

19. The method of claim 18, wherein Rₐ is selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, R_{b}-, and R_{b}-alkoxy; and
(b) alkoxycarbonylalkylamino, R_{b}-alkoxy-, cyano, substituted or unsubstituted heterocyclyl, and hydroxy.

20. The method of claim 19, wherein Rₐ is C₂₋₅ alkyl that is substituted with one or more substituents selected from the group consisting of amino, monoalkylamino, and dialkylamino.

21. The method of claim 14, wherein R₅ is 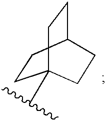
wherein said R₅ is either unsubstituted or substituted with one or more Rₐ groups selected from the group consisting of:
(a) C₁₋₆ alkyl, C₂₋₆ alkenyl, or C₂₋₆ alkynyl; wherein said alkyl, alkenyl, or alkynyl group is each either unsubstituted or substituted with one or more substituents selected from the group consisting of amino, monoalkylamino, dialkylamino, substituted or unsubstituted heterocyclylaminocarbonyl, (amino) (R_{b}) acylhydrazinylcarbonyl-, (amino) (R_{b}) acyloxycarboxy-, (hydroxy)(carboalkoxy)alkylcarbamoyl, acyloxy, aldehydo, alkenylsulfonylamino, alkoxy, alkoxycarbonyl, alkylaminoalkylamino, dialkylaminoalkylamino, alkylphosphono, alkylsulfonylamino, carbamoyl, R_{b}-, R_{b}-alkoxy-, R_{b}-alkylamino-, cyano, cyanoalkylcarbamoyl, cycloalkylamino, dialkylphosphoho, haloalkylsulfonylamino, heterocyclylalkylamino, heterocyclylcarbamoyl, hydroxy, hydroxyalkylsulfonylamino, oximino, phosphono, substituted or unsubstituted aralkylamino, substituted or unsubstituted arylcarboxyalkoxycarbonyl, substituted or unsubstituted heteroarylsulfonylamino, substituted or unsubstituted heterocyclyl, thiocarbamoyl, and trifluoromethyl; and
(b) (alkoxycarbonyl)aralkylcarbamoyl, aldehydo, alkenoxy, alkenylsulfonylamino, alkoxy, alkoxycarbonyl, alkylcarbamoyl, alkoxycarbonylamino, alkoxycarbonylalkylamino, alkylsulfonylamino, alkylsulfonyloxy, amino, aminoalkylaralkylcarbamoyl, aminoalkylcarbamoyl, aminoalkylheterocyclylalkylcarbamoyl, aminocycloalkylalkylcycloalkylcarbamoyl,
aminocycloalkylcarbamoyl, aralkoxycarbbnylamino, arylheterocyclyl, aryloxy, arylsulfonylamino, arylsulfonyloxy, carbamoyl, carbonyl, R_{b}-, R_{b}-alkoxy-, R_{b}-alkylthio-, R_{b}-alkyl(alkyl)amino-, R_{b}-alkyl(alkyl)carbamoyl-, R_{b}-alkylamino-, R_{b}-alkylcarbamoyl-, R_{b}-alkylsulfonyl-, R_{b}-alkylsulfonylamino, R_{b}-alkylthio, R_{b}-heterocyclylcarbonyl, aminoalkylaminocarbonyl, dialkylaminoalkylamino, alkylaminoalkylamino, cyano, cycloalkylamino, dialkylamino alkylcarbamoyl, halogen, heterocyclylalkylamino, hydroxy, oximino, phosphate, substituted or unsubstituted aralkylamino, substituted or unsubstituted heterocyclyl, substituted or unsubstituted heterocyclylsulfonylamino, sulfoxyacylamino, and thiocarbamoyl.

22. The method of claim 21, wherein Rₐ is selected from the group consisting of:
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, R_{b}-, and R_{b}-alkoxy-; and
(b) alkoxycarbonylalkylamino, R_{b}-alkoxy-, cyano, substituted or unsubstituted heterocyclyl, and hydroxy.

23. The method of claim 21, wherein Rₐ is selected from the group consisting of:
(a) C₁₋₄ alkyl or C₂₋₄ alkenyl, each of which is unsubstituted or substituted with one or more substituents selected from the group consisting of amino, monoalkylamino, dialkylamino, substituted or unsubstituted heterocyclylaminocarbonyl, substituted or unsubstituted heterocyclyl, and R_{b}-; and
(b) R_{b}-alkoxy- and substituted heterocyclyl.

24. The method of claim 1, wherein:
each of R₁ and R₂ is propyl;
R₃ is hydrogen;
R₄ is a single bond;
R₅ is phenyl substituted with Rₐ, 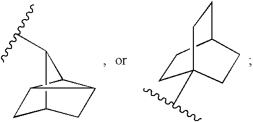
wherein said bicyclic or trycyclic group is optionally substituted with Rₐ;
Rₐ is selected from the group consisting of;
(a) C₁₋₆ alkyl or C₂₋₆ alkenyl, each of which is unsubstituted or substituted with one or more substituents selected from the group consisting of amino, monoalkylamino, dialkylamino, substituted or unsubstituted heterocyclylaminocarbonyl, R_{b}-, R_{b}-alkoxy-, and substituted or unsubstituted heterocyclyl; and
(c) alkoxycarbonylalkylamino, cyano, and hydroxy; each of X₁ and X₂ is O; and
X₃ is N.

25. The method of claim 1, wherein the compound of formula (I) is 3-[4-(2,6-dioxo-1,3-dipropyl-2,3,6,7-tetrahydro-1H-purin-8-yl)-bicyclo[2.2.2]oct-1-yl]-propionic acid.

26. The method of claim 1, wherein the ischemic event is selected from the group consisting of acute coronary syndrome, stroke, organ transplantation, kidney ischemia, shock, and organ transplantation surgery.

27. The method of claim 26, wherein the acute coronary syndrome is myocardial infarction.

28. The method of claim 1, wherein the A_{2b} adenosine receptor antagonist is administered within two days before or after the ischemic event.

29. The method of claim 28, wherein the A_{2b} adenosine receptor antagonist-is administered within two days after the ischemic event.

30. The method of claim 1, wherein mammal is a human.

31. The method of claim 1, wherein the compound of formula (I) exhibits an affinity for an A_{2b} adenosine receptor that is at least 10-fold greater than the affinity for an A₂ₐ adenosine receptor or an A₃ adenosine receptor.

32. The method of claim 31, wherein the compound of formula (I) further exhibits an affinity for an A₁ adenosine receptor that is at least 10-fold greater than the affinity for the A₂ₐ adenosine receptor or the A₃ adenosine receptor.

33. The method of claim 1, wherein the compound of formula (I) exhibits a Kᵢ value for an A_{2b} adenosine receptor below 500 nM.

34. The method of claim 1, wherein the compound of formula (I) exhibits a Kᵢ value for an A_{2b} adenosine receptor below 200 nM.

35. A method of treating a disease or disorder mediated by activation of an A_{2b} adenosine receptor comprising administering to a mammal in need thereof an effective amount of a compound of formula (I) according to claim 1.

36. A method of limiting tissue necrosis resulting from an ischemic event, comprising:
identifying a mammal that has undergone an ischemic event, or in which an ischemic event is imminent; and
administering a therapeutically effective or prophylactically effective amount of an A_{2b} adenosine receptor antagonist to the mammal within ten days before or after the ischemic event;
wherein the A_{2b} adenosine receptor antagonist is a compound of formula (I) according to claim 1.

37. A method of limiting infarction size following myocardial infarction, comprising:
identifying a mammal that has undergone myocardial infarction, or in which myocardial infarction is imminent; and
administering a therapeutically effective or prophylactically effective amount of an A_{2b} adenosine receptor antagonist to the mammal within ten days before or after the myocardial infarction;
wherein the A_{2b} adenosine receptor antagonist is a compound of formula (I) according to claim 1.
